(19) United States
(12) Patent Application Publication
(10) Pub. No.: US 2026/0224910 A1
(43) Pub. Date: Aug. 6, 2026

(54) LIVING BODY ATTACHMENT-TYPE LIGHT EMISSION DEVICE

(71) Applicant: SONY GROUP CORPORATION, TOKYO (JP)

(72) Inventors: HIROYUKI SUZUKI, KANAGAWA (JP); TATSUYA ICHIKAWA, KANAGAWA (JP)

(21) Appl. No.: 19/151,339

(22) PCT Filed: Jan. 26, 2024
(86) PCT No.: PCT/JP2024/002319

§ 371 Date: Jul. 28, 2025

(30) Foreign Application Priority Data
Feb. 8, 2023 (JP) 2023-017929

(51) Int. Cl. A61N 5/06

(52) U.S. Cl. CPC A61N 5/062; A61N 5/0601; A61N 2005/0628; A61N 2005/0652; A61N 2005/0666

(57) ABSTRACT

A living body attachment-type light emission device according to an embodiment of the present disclosure includes: a substrate; a light emitting unit that is provided on the substrate and emits light; and a flexible light guide plate that is joined to the substrate, guides the light emitted by the light emitting unit, and has a plurality of through holes.

### FIELD

The present disclosure relates to a living body attachment-type light emission device.

### BACKGROUND

Photodynamic therapy (PDT) is usually a method of injecting a photosensitive substance (photosensitizer) into a living body, emitting light having a certain wavelength to a target biological tissue, generating active oxygen from the photosensitive substance, and treating a lesion such as cancer or an infectious disease with the active oxygen. As a device used for this photodynamic therapy, a living body attachment-type light emission device attached to a living body has been developed. For example, Patent Literature 1 proposes a probe having a light source uniformly on an entire surface of a base material.

### CITATION LIST

### Patent Literature

Patent Literature 1: JP H11-507284 A

### SUMMARY

### Technical Problem

However, in the above-described probe, breathability and liquid permeability are poor, and biocompatibility is low. In addition, since a large number of light sources are present in a base material, flexibility is decreased, and a cost is increased.

Thus, the present disclosure provides a living body attachment-type light emission device capable of improving biocompatibility, improving flexibility, and reducing a cost.

### Solution to Problem

A living body attachment-type light emission device according to an embodiment of the present disclosure includes: a substrate; a light emitting unit that is provided on the substrate and emits light; and a flexible light guide plate that is joined to the substrate, guides the light emitted by the light emitting unit, and has a plurality of through holes. Note that, hereinafter, the living body attachment-type light emission device will be simply referred to as a light emission device.

### BRIEF DESCRIPTION OF DRAWINGS

FIG. 1 is a plan view illustrating a configuration example of a light emission device according to a first embodiment.

FIG. 2 is a cross-sectional view illustrating the configuration example of the light emission device according to the first embodiment.

FIG. 3 is a cross-sectional view illustrating a configuration example of a light emission device according to a modification example of the first embodiment.

FIG. 4 is a view illustrating an example of attachment to a living body of the light emission device according to the first embodiment.

FIG. 5 is a view illustrating a configuration example of an endoscopic device according to the first embodiment.

FIG. 6 is a perspective view illustrating a configuration example of a camera distal end portion of the endoscopic device according to the first embodiment.

FIG. 7 is a cross-sectional view illustrating a configuration example of a light emission device according to a

FIG. 8 is a cross-sectional view illustrating a configuration example of a light emission device according to a modification example of the second embodiment.

FIG. 9 is a cross-sectional view illustrating a configuration example of a light emission device according to a third embodiment.

FIG. 10 is a cross-sectional view illustrating a configuration example of a light emission device according to a modification example of the third embodiment.

FIG. 11 is a cross-sectional view illustrating a configuration example of a light emission device according to a fourth embodiment.

FIG. 12 is a cross-sectional view illustrating a configuration example of a light emission device according to a fifth embodiment.

FIG. 13 is a plan view illustrating a configuration example of a light emission device according to a sixth embodiment.

FIG. 14 is a cross-sectional view illustrating a configuration example of a light emission device according to a modification example of the sixth embodiment.

FIG. 15 is a side view illustrating the configuration example of the light emission device according to the modification example of the sixth embodiment.

FIG. 16 is a plan view illustrating a configuration example of a light emission device according to a seventh embodiment.

FIG. 17 is a view illustrating a configuration example of a light emission device according to an eighth embodiment.

FIG. 18 is a cross-sectional view illustrating a configuration example of a light emission device according to a ninth embodiment.

FIG. 19 is a cross-sectional view illustrating a configuration example of a light emission device according to a modification example of the ninth embodiment.

FIG. 20 is a cross-sectional view illustrating a configuration example of a light emission device according to a tenth embodiment.

FIG. 21 is a cross-sectional view illustrating a configuration example of a light emission device according to a modification example of the tenth embodiment.

FIG. 22 is a cross-sectional view illustrating a configuration example of a light emission device according to an eleventh embodiment.

FIG. 23 is a cross-sectional view illustrating a configuration example of a light emission device according to a twelfth embodiment.

FIG. 24 is a cross-sectional view illustrating a configuration example of a light emission device according to a first modification example of the twelfth embodiment.

FIG. 25 is a cross-sectional view illustrating a configuration example of a light emission device according to a second modification example of the twelfth embodiment.

FIG. 26 is a cross-sectional view illustrating a configuration example of a light emission device according to a third modification example of the twelfth embodiment.

FIG. 27 is a plan view illustrating a configuration example of a light emission device according to a thirteenth embodiment.

FIG. 28 is a cross-sectional view illustrating a configuration example of a light emission device according to a fourteenth embodiment.

FIG. 29 is a view for describing a light reaching range based on presence or absence of a protrusion portion according to the fourteenth embodiment.

FIG. 30 is a cross-sectional view illustrating a protrusion portion according to the fourteenth embodiment.

FIG. 31 is a cross-sectional view illustrating a configuration example of a light emission device according to a first modification example of the fourteenth embodiment.

FIG. 32 is a cross-sectional view illustrating a configuration example of a light emission device according to a second modification example of the fourteenth embodiment.

FIG. 33 is a cross-sectional view illustrating a configuration example of a light emission device according to a third modification example of the fourteenth embodiment.

FIG. 34 is a plan view illustrating a configuration example of a light emission device according to a fifteenth embodiment.

FIG. 35 is a graph illustrating a relationship between intensity of light absorption/scattering and a wavelength according to the fifteenth embodiment.

FIG. 36 is a cross-sectional view illustrating a configuration example of a light emission device according to a modification example of the fifteenth embodiment.

FIG. 37 is a cross-sectional view illustrating a configuration example of a light emission device according to a sixteenth embodiment.

### DESCRIPTION OF EMBODIMENTS

In the following, embodiments of the present disclosure will be described in detail on the basis of the drawings. The embodiments also include examples, modification examples, and the like. Note that a device, a method, and the like according to the present disclosure are not limited by the embodiments of the present disclosure. Furthermore, in each of the following embodiments, overlapped description is omitted by assignment of the same reference sign to parts that are basically the same.

Each of the following one or more embodiments can be implemented independently. On the other hand, at least a part of the following plurality of embodiments may be implemented by being appropriately combined with at least a part of other embodiments. The plurality of embodiments may include novel features different from each other. Thus, the embodiments can contribute to solving different objects or problems, and can exhibit different effects. Note that an effect in each of the embodiments is merely an example and is not limited, and there may be another effect.

The present disclosure will be described in the following order of items.
1. First embodiment
1-1. Configuration example of a light emission device
1-2. Modification example
1-3. Example of attachment to a living body
1-4. Configuration example of an endoscopic device
2. Second embodiment
2-1. Configuration example of a light emission device
2-2. Modification example
3. Third embodiment
3-1. Configuration example of a light emission device
3-2. Modification example
4. Fourth embodiment
4-1. Configuration example of a light emission device
5. Fifth embodiment
5-1. Configuration example of a light emission device
6. Sixth embodiment
6-1. Configuration example of a light emission device
6-2. Modification example
7. Seventh embodiment
7-1. Configuration example of a light emission device
8. Eighth embodiment
8-1. Configuration example of a light emission device
9. Ninth embodiment
9-1. Configuration example of a light emission device
9-2. Modification example
10. Tenth embodiment
10-1. Configuration example of a light emission device
10-2. Modification example
11. Eleventh embodiment
11-1. Configuration example of a light emission device
12. Twelfth embodiment
12-1. Configuration example of a light emission device
12-2. First modification example
12-3. Second modification example
12-4. Third modification example
13. Thirteenth embodiment
13-1. Configuration example of a light emission device
14. Fourteenth embodiment
14-1. Configuration example of a light emission device
14-2. First modification example
14-3. Second modification example
14-4. Third modification example
15. Fifteenth embodiment
15-1. Configuration example of a light emission device
15-2. Modification example
16. Sixteenth embodiment
16-1. Configuration example of a light emission device
17. Action and effect according to each embodiment
18. Other embodiments
19. Supplementary note

### 1. First Embodiment

### <1-1. Configuration Example of a Light Emission Device>

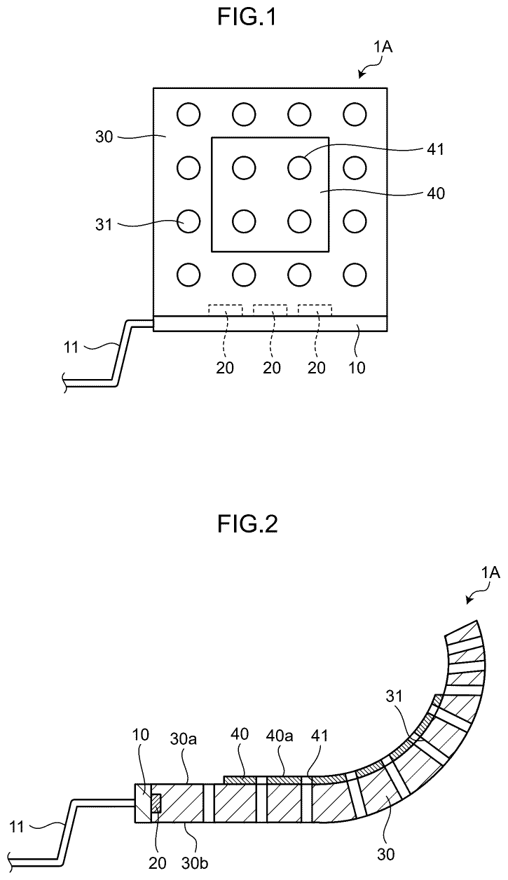

A configuration example of a light emission device 1A according to the first embodiment will be described with reference to FIG. 1 and FIG. 2. FIG. 1 is a plan view illustrating the configuration example of the light emission device 1A according to the first embodiment. FIG. 2 is a cross-sectional view illustrating the configuration example of the light emission device 1A according to the first embodiment.

As illustrated in FIG. 1 and FIG. 2, the light emission device 1A according to the first embodiment includes a substrate 10, a plurality of light emitting units 20, a flexible light guide plate 30, and a diffusion layer 40.

The substrate 10 includes, for example, a printed wiring board. The substrate 10 is formed in, for example, a rectangular shape extending in one direction in plan view. A lead wire 11 is connected to the substrate 10. The lead wire 11 is electrically connected to, for example, a printed wiring line (not illustrated) provided on the substrate 10. Note that although various substrates are used as the substrate 10, a flexible substrate may be used, for example.

Each of the light emitting units 20 is provided on the substrate 10 and emits light. These light emitting units 20 are, for example, arranged in a line in an extending direction of the substrate 10, and emit light in a direction along a surface 30a of the flexible light guide plate 30. The surface 30a of the flexible light guide plate 30 is an upper surface in FIG. 2 and is a surface on a living body attachment side of the flexible light guide plate 30. Each of the light emitting units 20 is electrically connected to, for example, the printed wiring line (not illustrated) provided on the substrate 10, and power is supplied to these light emitting units 20 via the printed wiring line and the lead wire 11 of the substrate 10. The number of the light emitting units 20 is not specifically limited, and may be one or two or more. As each of the light emitting units 20, for example, a light source such as a light emitting diode (LED), an organic light emitting diode (OLED), or a VCSEL is used.

The flexible light guide plate 30 has flexibility and is joined to the substrate 10. Specifically, an end surface of the flexible light guide plate 30 and a surface of the substrate 10 on a side of each of the light emitting units 20 are joined. The flexible light guide plate 30 guides the light emitted by each of the light emitting units 20 to the entire surface. In addition, the flexible light guide plate 30 has a plurality of through holes 31. Each of these through holes 31 is, for example, a mesh portion, but is not limited thereto. The flexible light guide plate 30 is formed of, for example, polydimethylsiloxane (PDMS), polyethylene glycol (PEG), polylactic acid (PLA), or the like.

The diffusion layer 40 is provided on the surface 30a of the flexible light guide plate 30, and diffuses the light guided by the flexible light guide plate 30. In addition, the diffusion layer 40 has a plurality of through holes 41 respectively connected to the through holes 31. Each of these through holes 41 is, for example, a mesh portion, but is not limited thereto. For example, the diffusion layer 40 is formed by mixing of nanoparticles (for example, high refractive materials such as TiO2 and ZrO2) with the material of the flexible light guide plate 30, or formed by execution of surface processing of making a surface of an object uneven on the surface 30a of the flexible light guide plate 30. Note that in a case where the nanoparticles are used, a particle size is preferably about 25 to 100 nm.

Here, as illustrated in FIG. 1, each of the flexible light guide plate 30 and the diffusion layer 40 is formed in, for example, a square shape in the plan view. An area of the diffusion layer 40 is smaller than an area of the flexible light guide plate 30, for example. In the example of FIG. 1, the diffusion layer 40 is positioned at a center of the flexible light guide plate 30, but is not limited thereto. The diffusion layer 40 functions as a determination unit that determines a light emitting region that emits light. It is possible to change an area and position of the light emitting region by changing the area and position of the diffusion layer 40. Light diffusivity of a part of the flexible light guide plate 30 is improved by the diffusion layer 40, and the light emission device 1A can output light only in an assumed area.

Each of the through holes 31 of the flexible light guide plate 30 and each of the through holes 41 of the diffusion layer 40 enable exchange of gas, liquid, a chemical substance, and the like with a living body (for example, a lesion such as a tumor). That is, the through holes 31 and the through holes 41 communicate with each other, and the gas, the liquid, the chemical substance, and the like can pass through the through holes 31 and 41 from a surface 30b of the flexible light guide plate 30 and reach the surface 30a of the flexible light guide plate 30. The surface 30b of the flexible light guide plate 30 is a lower surface in FIG. 2, and is a surface on an opposite side of the living body attachment side of the flexible light guide plate 30.

Note that in the example of FIG. 1, the through holes 31 of the flexible light guide plate 30 and the through holes 41 of the diffusion layer 40 are aligned in a matrix. These through holes 31 and 41 do not need to be specifically aligned, but are preferably arranged in a matrix for uniformization of a light emission distribution. In addition, since the light emission distribution and light emission efficiency are improved, it is preferable that rows and columns have the same pitch. A shape of the holes is not limited to a circular shape, but is preferably a circle. In addition, the through holes 31 are preferably absent in a vicinity of (for example, proximity of) each of the light emitting units 20 since light emission efficiency is improved. Note that the through holes 31 may not be formed over the entire surface of the flexible light guide plate 30, the through holes 41 may not be formed over the entire surface of the diffusion layer 40, and the through holes 31 and 41 may be formed in a part of the surfaces. However, in order to improve breathability and liquid permeability, it is preferable that the through holes 31 and 41 are formed in the entire surfaces.

According to the light emission device 1A in a manner described above, since the flexible light guide plate 30 has the through holes 31 and the diffusion layer 40 has the through holes 41 respectively connected to the through holes 31, the breathability and the liquid permeability of the light emission device 1A are improved. As a result, biocompatibility can be improved. Furthermore, the substrate 10 includes only a several (three in the example of FIG. 1) light emitting units 20, and the light emitting units 20 are not present in the flexible light guide plate 30. Thus, as compared with a case where a large number of light emitting units 20 are present on the entire surface of the light emission device 1A as in related art, the number of light emitting units 20 can be reduced, flexibility can be improved, and a cost can be reduced.

In addition, all the light emitting portions that emit light to the outside can be freely bent since being flexible and there is no wiring line, device, and the like. Accordingly, the flexibility can be further improved. In addition, the light emitting units 20 and the like are covered with an end portion of the flexible light guide plate 30, and are prevented from directly touching the living body, gas, liquid, and the like. In addition, it is also possible to separate only a light guide plate portion such as the flexible light guide plate 30, and reuse the substrate 10 and each of the light emitting units 20. Note that the light emission device 1A may be disposable.

In addition, for example, as compared with a case where a laser device that emits light to a tumor via an optical fiber is used, the light emission device 1A is attached and fixed to the living body. Thus, it is possible to realize stabilization of emission and improvement of light utilization efficiency. Furthermore, by adjusting the size and position of the diffusion layer 40, the area (light emission area) and position of the light emitting region can be easily changed. In addition, a therapeutic effect in a depth direction can be improved when time is taken. In addition, treatment can be performed anywhere the light emission device 1A can be attached.

### <1-2. Modification Example>

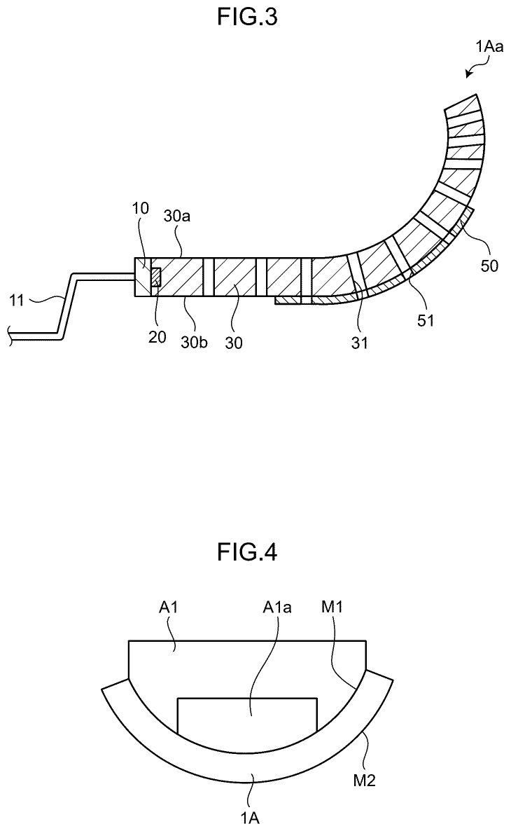

A configuration example of a light emission device 1Aa according to a modification example of the first embodiment will be described with reference to FIG. 3. FIG. 3 is a cross-sectional view illustrating the configuration example of the light emission device 1Aa according to the modification example of the first embodiment. In the modification example of the first embodiment, a point different from the first embodiment will be described.

As illustrated in FIG. 3, the light emission device 1Aa according to the modification example of the first embodiment includes the components according to the first embodiment, and includes a reflection layer 50 instead of the diffusion layer 40. The reflection layer 50 is provided on a surface 30b of a flexible light guide plate 30, and reflects light guided by the flexible light guide plate 30. In addition, the reflection layer 50 has a plurality of through holes 51 respectively connected to through holes 31. Each of these through holes 51 is, for example, a mesh portion, but is not limited thereto. Since the reflection layer 50 has the through holes 51, breathability and liquid permeability of the light emission device 1Aa are improved. Note that the reflection layer 50 is formed by utilization of, for example, a high reflectance material (for example, a metal such as Al, a highly reflective silicone, or the like), an uneven pattern, a polymer, or nanoparticles.

The reflection layer 50 is formed in, for example, a square shape in plan view. An area of the reflection layer 50 is smaller than an area of the flexible light guide plate 30, for example. For example, the reflection layer 50 is positioned at a center of the flexible light guide plate 30, but is not limited thereto. The reflection layer 50 functions as a determination unit that determines a light emitting region that emits light. It is possible to change an area and a position of the light emitting region by changing the area and position of the reflection layer 50. Reflectance of a part of the flexible light guide plate 30 is improved by the reflection layer 50, and the light emission device 1Aa can output light only in an assumed area.

According to the light emission device 1Aa in a manner described above, an effect similar to that of the first embodiment can be acquired. For example, since the flexible light guide plate 30 has the through holes 31 and the reflection layer 50 has the through holes 51 respectively connected to the through holes 31, breathability and liquid permeability of the light emission device 1Aa are improved. As a result, biocompatibility can be improved. Furthermore, the substrate 10 includes only a several light emitting units 20, and the light emitting units 20 are not present in the flexible light guide plate 30. Thus, as compared with a case where a large number of light emitting units 20 are present on the entire surface of the light emission device 1Aa as in the related art, the number of light emitting units 20 can be reduced, the flexibility can be improved, and a cost can be reduced.

### <1-3. Example of Attachment to a Living Body>

An example of attachment to a living body of the light emission device 1A according to the first embodiment will be described with reference to FIG. 4. FIG. 4 is a view illustrating the example of the attachment to the living body of the light emission device 1A according to the first embodiment.

As illustrated in FIG. 4, the light emission device 1A is positioned in such a manner as to face a tumor A1a of a living body A1, and is attached to the living body A1. The tumor A1a is an example of a lesion. For example, the tumor A1a is present in the living body A1, and the light emission device 1A is attached to a desired position inside the living body A1. A living body attachment surface M1 of the light emission device 1A is a surface attached to the living body A1, and a living body non-attachment surface M2 of the light emission device 1A is a surface on an opposite side of the living body attachment surface M1 and is a surface not attached to the living body A1. The living body attachment surface M1 of the light emission device 1A includes the surface 30a of the flexible light guide plate 30 and a surface 40a of the diffusion layer 40 illustrated in FIG. 2. The surface 40a of the diffusion layer 40 is an upper surface in FIG. 2, and is a surface on the living body attachment side of the diffusion layer 40. Note that the living body attachment surface M1 of the light emission device 1Aa includes only the surface 30a of the flexible light guide plate 30.

In order to place the light emission device 1A or the light emission device 1Aa in the above-described manner inside the living body A1, for example, an endoscopic device, a catheter (such as a balloon catheter), or the like can be used. Note that in the endoscopic device, the catheter, or the like, a stent can be used in order to place the light emission device 1A or the light emission device 1Aa inside the living body A1 (details will be described later).

### <1-4. Configuration Example of an Endoscopic Device>

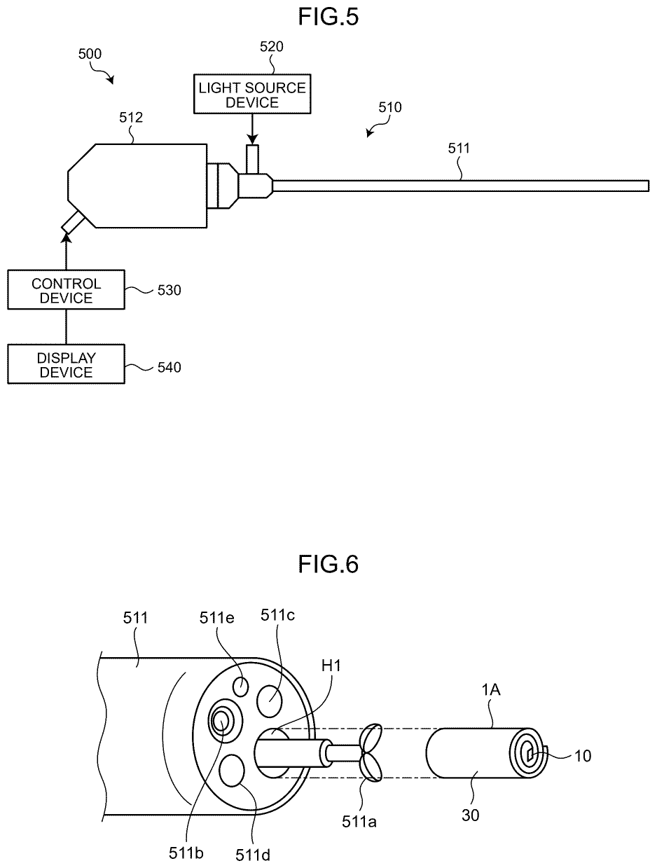

A configuration example of an endoscopic device 500 according to the first embodiment will be described with reference to FIG. 5 and FIG. 6. FIG. 5 is a view illustrating the configuration example of the endoscopic device 500 according to the first embodiment. FIG. 6 is a perspective view illustrating a configuration example of a distal end portion of a camera 510 according to the first embodiment.

### (Overall Configuration of the Endoscopic Device)

As illustrated in FIG. 5, the endoscopic device 500 includes the camera 510, a light source device 520, a control device 530, and a display device 540. The camera 510 includes an optical system 511 and a camera head 512.

The optical system 511 is formed in, for example, a soft or hard elongated shape, and is inserted into the living body. The optical system 511 guides light from the light source device 520 to a subject and guides light reflected by the subject to the camera head 512. The optical system 511 includes a light source optical system, an imaging optical system, and the like.

The camera head 512 captures a subject image collected by the optical system 511 under control of the control device 530, and outputs an imaging signal acquired by the imaging. The camera head 512 includes, for example, an imaging unit (not illustrated), and images various subjects in the body by the imaging unit. The imaging unit is realized by, for example, an image sensor capable of color photographing, or the like.

The light source device 520 supplies illumination light such as white light or near-infrared light to the optical system 511. The white light is, for example, illumination light for illumination inside the living body, and the near-infrared light is illumination light for special observation. The light source device 520 may be, for example, a light source device capable of continuously or gradually changing a wavelength and an emission direction of emitted light.

The control device 530 processes the imaging signal input from the camera head 512, outputs an image signal to the display device 540, and integrally controls operations of the camera head 512, the light source device 520, the display device 540, and the like.

The display device 540 displays an image generated by the control device 530 under the control of the control device 530. The display device 540 is realized by, for example, a liquid crystal display, an organic electro-luminescence (EL) display, or the like. Note that the display device 540 may be a device integrated with the control device 530, or may be a device separate from the control device 530.

### (Distal End Portion of the Camera)

As illustrated in FIG. 6, forceps 511a, an objective lens 511b, a plurality of light guides 511c and 511d, and a nozzle 511e are provided at a distal end portion of the optical system 511. The forceps 511a are a surgical tool for gripping and pulling a target object, and are configured to be able to enter and exit from a forceps port H1. The objective lens 511b is a lens for imaging, each of the light guides 511c and 511d emits light, and the nozzle 511e sends out water, air, or the like as necessary.

For example, the light emission device 1A is rolled into a scroll shape in which an end portion on a side of the substrate 10 of the light emission device 1A is located at a center, and is inserted into the forceps port H1 of the distal end portion of the optical system 511. In this case, when the distal end portion of the optical system 511 reaches a target position in the body, the forceps 511a are taken out from the forceps port H1. At this time, the scroll-shaped light emission device 1A comes out from the forceps port H1 together with the forceps 511a. Then, the scroll-shaped light emission device 1A is elastically unfolded and attached to a desired position in the body automatically or by the forceps 511a.

### 2. Second Embodiment

### <2-1. Configuration Example of a Light Emission Device>

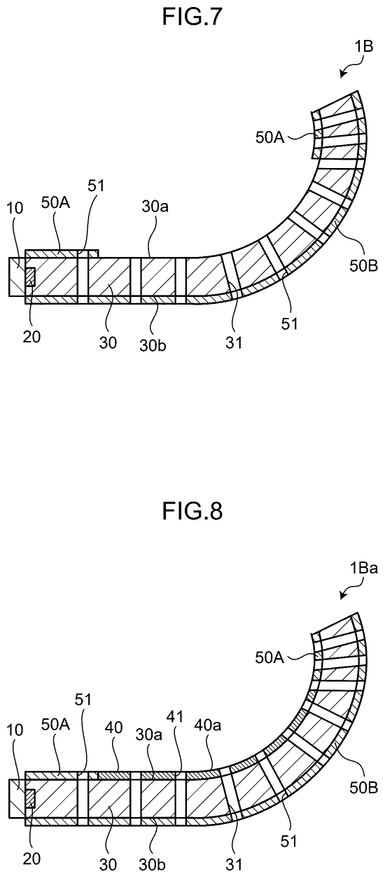

A configuration example of a light emission device 1B according to the second embodiment will be described with reference to FIG. 7. FIG. 7 is a cross-sectional view illustrating a configuration example of a light emission device 1B according to the second embodiment. In the second embodiment, a point different from a modification example of the first embodiment will be described.

As illustrated in FIG. 7, the light emission device 1B according to the second embodiment includes components according to the modification example of the first embodiment, and includes reflection layers 50A and 50B instead of the reflection layer 50. For example, the reflection layer 50A is provided in the outer peripheral region of the surface 30a of the flexible light guide plate 30, and the reflection layer 50B is provided in the entire region of the surface 30b of the flexible light guide plate 30. Each of the reflection layers 50A and 50B reflects the light guided by the flexible light guide plate 30. In addition, each of the reflection layers 50A and 50B has a plurality of through holes 51. Each of these through holes 51 is, for example, a mesh portion, but is not limited thereto. Since each of the reflection layers 50A and 50B has the through holes 51, breathability and liquid permeability of the light emission device 1B are improved.

The reflection layer 50A is formed in, for example, a square shape in the plan view. In the surface 30a of the flexible light guide plate 30, the region surrounded by the ring-shaped reflection layer 50A is positioned, for example, at the center of the flexible light guide plate 30, but is not limited thereto. Since the region surrounded by the ring-shaped reflection layer 50A is a light emitting region, each of the reflection layers 50A and 50B functions as a determination unit that determines the light emitting region of the flexible light guide plate 30. It is possible to change the area and position of the light emitting region by changing the area and position of the region surrounded by the ring-shaped reflection layer 50A. Reflectance of a part of the flexible light guide plate 30 is improved by each of the reflection layers 50A and 50B, and the light emission device 1B can output light only in the assumed area.

According to the light emission device 1B in a manner described above, an effect similar to that of the first embodiment can be acquired. Furthermore, since the light utilization efficiency is improved, the intensity of light emitted from the light emitting region can be increased. Note that a light control layer (such as meta-surface or the like) that controls light may be provided instead of the reflection layer 50B. Accordingly, light emission efficiency can be improved.

### <2-2. Modification Example>

A configuration example of a light emission device 1Ba according to a modification example of the second embodiment will be described with reference to FIG. 8. FIG. 8 is a cross-sectional view illustrating a configuration example of the light emission device 1Ba according to the modification example of the second embodiment. In the modification example of the second embodiment, a point different from the second embodiment will be described.

As illustrated in FIG. 8, the light emission device 1Ba according to the modification example of the second embodiment includes a diffusion layer 40 in addition to the components according to the second embodiment. The diffusion layer 40 is provided on the surface 30a of the flexible light guide plate 30. The diffusion layer 40 is provided in a region surrounded by the annular reflection layer 50A on the surface 30a of the flexible light guide plate 30.

According to the light emission device 1Ba in a manner described above, an effect similar to those of the first embodiment and the second embodiment can be acquired. Furthermore, since the light utilization efficiency is improved as compared with the second embodiment, the intensity of light emitted from the light emitting region that is the diffusion layer 40 can be increased.

### 3. Third Embodiment

### <3-1. Configuration Example of a Light Emission Device>

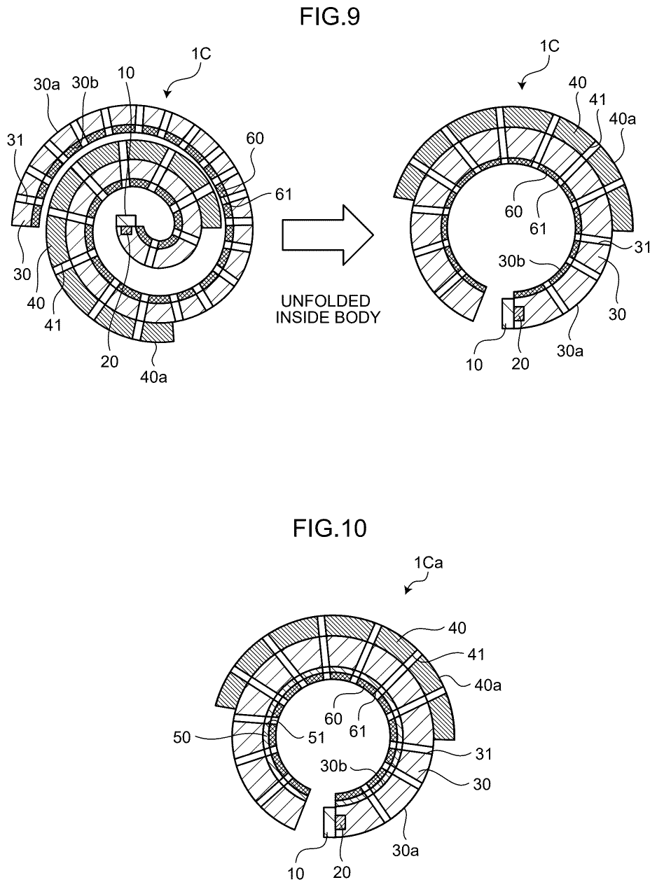

A configuration example of a light emission device 1C according to the third embodiment will be described with reference to FIG. 9. FIG. 9 is a cross-sectional view illustrating a configuration example of a light emission device 1C according to the third embodiment. In the third embodiment, a point different from the first embodiment will be described.

As illustrated in FIG. 9, a light emission device 1C according to the third embodiment includes a non-adhesive layer 60 in addition to the components according to the first embodiment. The non-adhesive layer 60 has non-adhesiveness and is provided on the entire surface of the surface 30b of the flexible light guide plate 30. The non-adhesive layer 60 is formed by, for example, coating with fluorine or the like, a polymer layer containing fluorine or the like, or plasma treatment of fluorine or the like. Note that the non-adhesive layer 60 may have translucency.

The light emission device 1C is, for example, rolled into a scroll shape in which an end portion on a side of a substrate 10 of the light emission device 1C is located at a center. The scroll-shaped light emission device 1C is unfolded at a desired position in a body. In the scroll-shaped light emission device 1C, there is a case where portions of the flexible light guide plate 30 adhere to each other and the unfolding becomes difficult. Thus, when the non-adhesive layer 60 is provided on the surface 30b of the flexible light guide plate 30, the non-adhesive layer 60 is present on an opposite side of a diffusion layer 40 with the flexible light guide plate 30 being interposed therebetween. As a result, since it is possible to control adhesion between the portions of the flexible light guide plate 30, it is possible to make it easy to unfold the scroll-shaped light emission device 1C.

According to the light emission device 1C in a manner described above, an effect similar to that of the first embodiment can be acquired. Furthermore, the non-adhesive layer 60 is present on the opposite side of the diffusion layer 40 with the flexible light guide plate 30 being interposed therebetween, and it is possible to control adhesion between the portions of the flexible light guide plate 30 in the scroll-shaped light emission device 1C. Thus, the scroll-shaped light emission device 1C can be easily unfolded.

### <3-2. Modification Example>

A configuration example of a light emission device 1Ca according to a modification example of the third embodiment will be described with reference to FIG. 10. FIG. 10 is a cross-sectional view illustrating the configuration example of the light emission device 1Ca according to the modification example of the third embodiment. In the modification example of the third embodiment, a point different from the third embodiment will be described.

As illustrated in FIG. 10, the light emission device 1Ca according to the modification example of the third embodiment includes a reflection layer 50 in addition to the components according to the third embodiment. The reflection layer 50 is provided between a flexible light guide plate 30 and a non-adhesive layer 60. That is, the non-adhesive layer 60 is laminated on the reflection layer 50 on the flexible light guide plate 30.

According to the light emission device 1Ca in a manner described above, an effect similar to those of the first embodiment and the third embodiment can be acquired. For example, the non-adhesive layer 60 is present on an opposite side of the diffusion layer 40 with the flexible light guide plate 30 being interposed therebetween, and it becomes possible to control adhesion between portions of the flexible light guide plate 30 in the scroll-shaped light emission device 1Ca. Thus, the scroll-shaped light emission device 1Ca can be easily unfolded.

### 4. Fourth Embodiment

### <4-1. Configuration Example of a Light Emission Device>

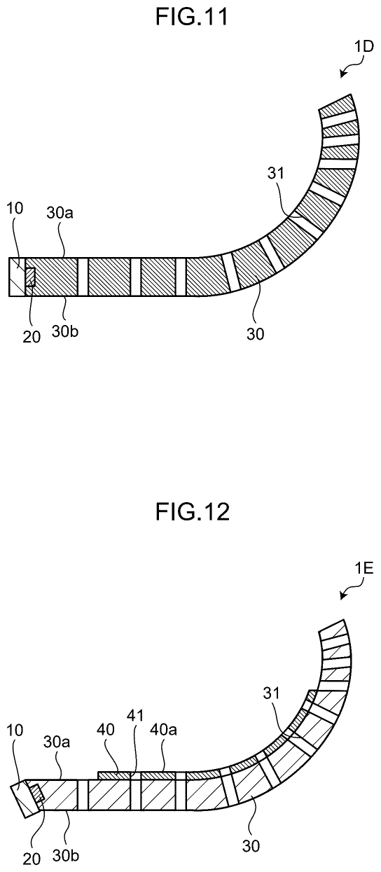

A configuration example of a light emission device 1D according to the fourth embodiment will be described with reference to FIG. 11. FIG. 11 is a cross-sectional view illustrating the configuration example of the light emission device 1D according to the fourth embodiment. In the fourth embodiment, a point different from the first embodiment will be described.

As illustrated in FIG. 11, the light emission device 1D according to the fourth embodiment does not include the diffusion layer 40 among the components according to the first embodiment, and a flexible light guide plate 30 has a function of diffusing light emitted by each light emitting unit 20 (light diffusion function) in addition to a function of guiding light. For example, nanoparticles (for example, high refractive materials such as TiO2 and ZrO2) are mixed in the flexible light guide plate 30 in order to cause the function of diffusing light. Note that since an entire surface of the flexible light guide plate 30 shines, a reflection layer 50 or the like may be used in a case where a light emitting region is limited.

According to the light emission device 1D in a manner described above, an effect similar to that of the first embodiment can be acquired. Furthermore, since the diffusion layer 40 can be made unnecessary as compared with the first embodiment, the configuration can be simplified. Note that in a case where a low-diffusion light source such as a laser is used as the light emitting unit 20, the configuration of the light emission device 1D is effective.

### 5. Fifth Embodiment

### <5-1. Configuration Example of a Light Emission Device>

A configuration example of a light emission device 1E according to the fifth embodiment will be described with reference to FIG. 12. FIG. 12 is a cross-sectional view illustrating the configuration example of the light emission device 1E according to the fifth embodiment. In the fifth embodiment, a point different from the first embodiment will be described.

As illustrated in FIG. 12, the light emission device 1E according to the fifth embodiment includes the components according to the first embodiment, and each light emitting unit 20 is provided to emit light toward a surface 30a of a flexible light guide plate 30. That is, each of the light emitting units 20 is located obliquely with respect to the surface 30a of the flexible light guide plate 30.

Here, although a thickness of the flexible light guide plate 30 covering the light emitting units 20 depends on a length of the light emitting units 20 in a short direction of the substrate 10 (length in a vertical direction in FIG. 2) in the first embodiment, a thickness of the flexible light guide plate 30 covering the light emitting units 20 can be reduced in the second embodiment since each of the light emitting units 20 is located obliquely with respect to the surface 30a of the flexible light guide plate 30. As a result, since flexibility of the light emission device 1E is improved, the light emission device 1E can be easily rolled into the body.

According to the light emission device 1E in a manner described above, an effect similar to that of the first embodiment can be acquired. Furthermore, since the flexible light guide plate 30 can be thinned as compared with the first embodiment, flexibility of the light emission device 1E can be improved.

### 6. Sixth Embodiment

### <6-1. Configuration Example of a Light Emission Device>

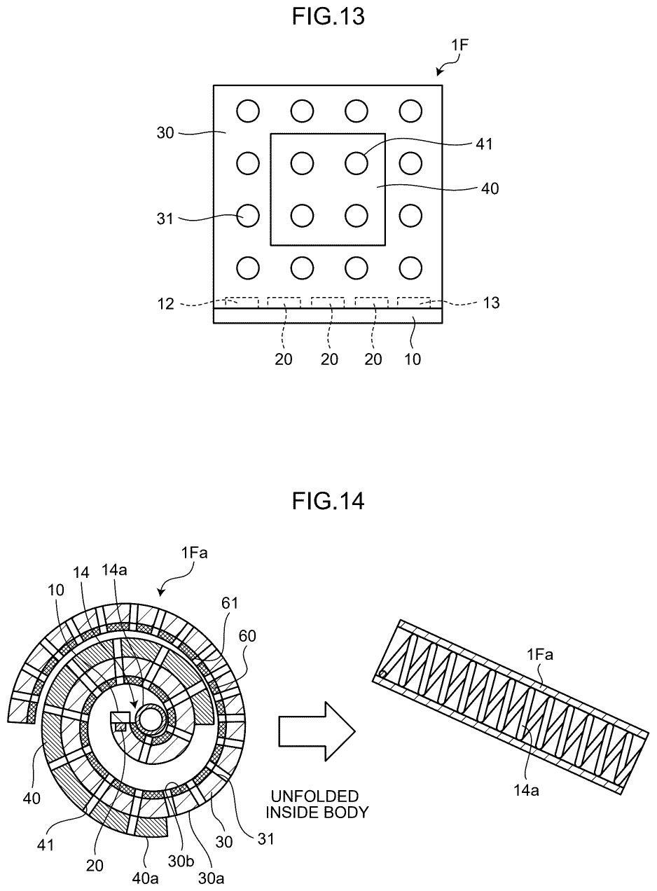

A configuration example of a light emission device 1F according to the sixth embodiment will be described with reference to FIG. 13. FIG. 13 is a plan view illustrating the configuration example of the light emission device 1F according to the sixth embodiment. In the sixth embodiment, a point different from the first embodiment will be described.

As illustrated in FIG. 13, the light emission device 1F according to the sixth embodiment includes a power supply 12 and a circuit 13 for communication or control in addition to the components according to the first embodiment. Note that the circuit 13 is provided as necessary.

The power supply 12 and the circuit 13 are provided on a substrate 10, and are electrically connected to, for example, a printed wiring line (not illustrated) of the substrate 10. The power supply 12 supplies power to each light emitting unit 20. As the power supply 12, a battery having high biocompatibility, for example, a battery such as an all-solid-state battery is used. The circuit 13 performs control related to communication or control. As the circuit 13, for example, an integrated circuit (IC) for communication or current control is used. Since being covered with a flexible light guide plate 30, the power supply 12 and the circuit 13 do not touch a living body and safety can be improved. In addition, by providing the power supply 12 and the circuit 13 on the substrate 10, it is possible to realize downsizing of the light emission device 1F.

According to the light emission device 1F in a manner described above, an effect similar to that of the first embodiment can be acquired. Furthermore, since the light emission device 1F includes the power supply 12, it is possible to drive the light emission device 1F in a stand-alone manner. Furthermore, in a case where communication or control (such as current control) is necessary, the light emission device 1F can deal with necessary communication or control since the circuit 13 for communication or control is included.

### <6-2. Modification Example>

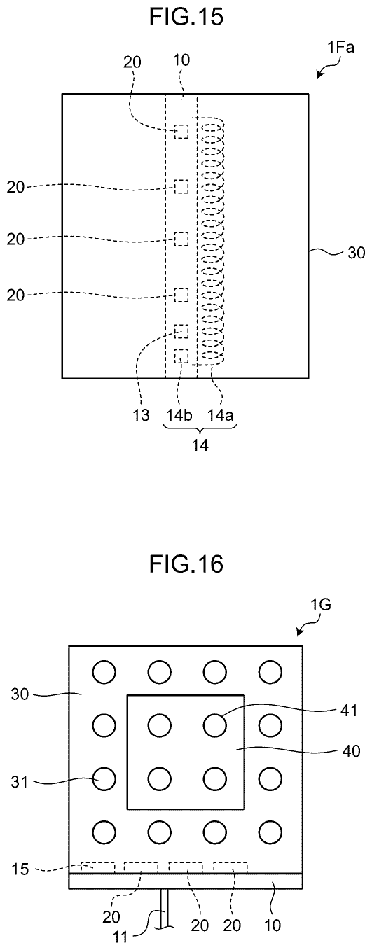

A configuration example of a light emission device 1Fa according to a modification example of the sixth embodiment will be described with reference to FIG. 14 and FIG. 15. FIG. 14 is a cross-sectional view illustrating the configuration example of the light emission device 1Fa according to the modification example of the sixth embodiment. FIG. 15 is a side view illustrating the configuration example of the light emission device 1Fa according to the modification example of the sixth embodiment. In the modification example of the sixth embodiment, a point different from the sixth embodiment will be described.

As illustrated in FIG. 14 and FIG. 15, the light emission device 1Fa according to the modification example of the sixth embodiment has the components according to the sixth embodiment, and includes a wireless power feeding unit 14 instead of the power supply 12. The wireless power feeding unit 14 includes a coil 14a and a capacitor 14b. Thus, the light emission device 1Fa is driven by a wireless power feeding method. The light emission device 1Fa is applicable to, for example, a ductal system such as a bile duct.

Both ends of the coil 14a are fixed to the substrate 10. The coil 14a and the capacitor 14b function as a resonator and are set to resonate with an applied magnetic field. The coil 14a, the capacitor 14b, and each of the light emitting units 20 on the substrate 10 are electrically connected by the printed wiring line (not illustrated) on the substrate 10. When a magnetic field is emitted to an inside of the coil 14a, the light emitting units 20 emit light by wireless power feeding. Since the both ends of the coil 14a are fixed to the substrate 10, a diameter of the coil 14a can be increased in accordance with unfolding of the light emission device 1Fa.

The light emission device 1Fa is rolled into a scroll shape, for example, in a state in which the diameter of the coil 14a both ends of which are fixed to the substrate 10 is small, that is, in a state in which the coil 14a is extended. The light emission device 1Fa is put into the body in the state of being rolled in the scroll shape. When the light emission device 1Fa is unfolded, a length of the coil 14a is reduced in accordance with the unfolding, and the diameter of the coil 14a is increased and expanded to a diameter close to a size of a tube to which the light emission device 1Fa is fixed.

According to the light emission device 1Fa in a manner described above, an effect similar to those of the first embodiment and the sixth embodiment can be acquired. Furthermore, since including the wireless power feeding unit 14, the light emission device 1Fa can be driven by the wireless power feeding method.

### 7. Seventh Embodiment

### <7-1. Configuration Example of a Light Emission Device>

A configuration example of a light emission device 1G according to the seventh embodiment will be described with reference to FIG. 16. FIG. 16 is a plan view illustrating the configuration example of the light emission device 1G according to the seventh embodiment. In the seventh embodiment, a point different from the first embodiment will be described.

As illustrated in FIG. 16, the light emission device 1G according to the seventh embodiment includes a sensor 15 in addition to the components according to the first embodiment. The sensor 15 detects a device state or a biological state. As the sensor 15, for example, a sensor that detects heat, light, or current is used. The sensor 15 detects a state of light emitting units 20 or estimates a state of a diffusion layer 40, for example, as the device state. Thus, the light emission device 1G can be driven under a condition that biological safety is reliably maintained. In addition, the sensor 15 detects, for example, light emission of a photosensitizer irradiated with blue light or the like as the biological state, and detects a state of a lesion such as cancer or tissue from the detection result (optical cancer diagnosis). The circuit 13 adjusts light emission intensity of the light emission device 1G according to, for example, the detected state of the cancer or tissue.

Note that the light emission device 1G may, for example, exchange sensor information with an external device via a lead wire 11 or the like, and control each of the light emitting units 20 by the external device beyond the lead wire 11. Alternatively, as in the sixth embodiment, a circuit 13 for communication or control may be included to control each of the light emitting units 20.

According to the light emission device 1G in a manner described above, an effect similar to that of the first embodiment can be acquired. Furthermore, since it becomes possible to detect the device state or the biological state and give feedback to the driving condition of each of the light emitting units 20 or the like, improvement of safety or a therapeutic effect can be realized.

### 8. Eighth Embodiment

### <8-1. Configuration Example of a Light Emission Device>

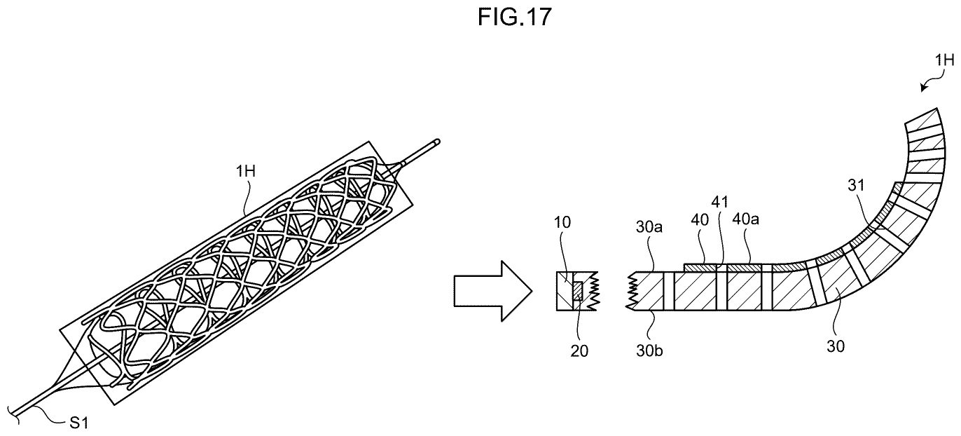

A configuration example of a light emission device 1H according to the eighth embodiment will be described with reference to FIG. 17. FIG. 17 is a view (perspective view and cross-sectional view) illustrating the configuration example of the light emission device 1H according to the eighth embodiment. In the eighth embodiment, a point different from the first embodiment will be described.

As illustrated in FIG. 17, the light emission device 1H according to the eighth embodiment has the components according to the first embodiment and is wound around a stent S1. That is, the light emission device 1H is used in combination with the stent S1. As a result, the light emission device 1H can be fixed and used in a ductal system such as a bile duct. Note that, for example, a normal stent diameter is about 8 to 10 mm, and a stent diameter of a gastrointestinal tract is about 2 to 3 mm.

A substrate 10 is formed to be separable from a flexible light guide plate 30. As a result, by separating the substrate 10 from the flexible light guide plate 30 and collecting the substrate 10 including each of light emitting units 20, it is also possible to leave a portion of the flexible light guide plate 30 in the body together with the stent S1. Since being formed of a biocompatible material, the flexible light guide plate 30 and a diffusion layer 40 may be left in the body. Note that by applying the sixth embodiment including the power supply 12 to the eighth embodiment, it is also possible to emit light for a long period.

According to the light emission device 1H in a manner described above, an effect similar to that of the first embodiment can be acquired. Furthermore, since the light-emitting device 1H is used in combination with the stent S1, the light emission device 1H can be fixed and used in the ductal system such as the bile duct.

### 9. Ninth Embodiment

### <9-1. Configuration Example of a Light Emission Device>

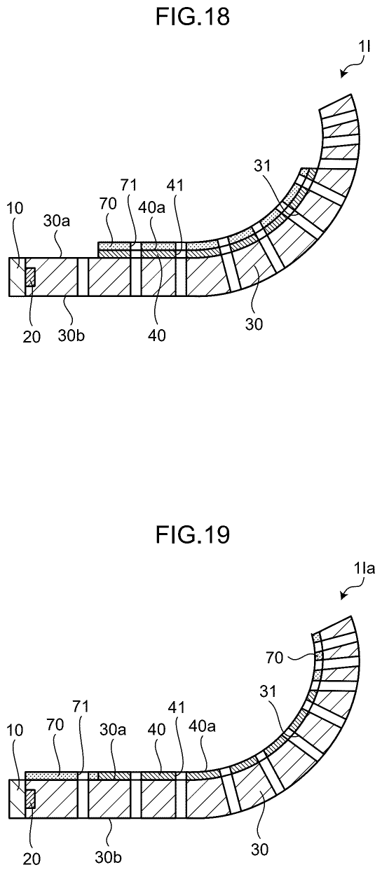

A configuration example of a light emission device 1I according to the ninth embodiment will be described with reference to FIG. 18. FIG. 18 is a cross-sectional view illustrating the configuration example of the light emission device 1I according to the ninth embodiment. In the ninth embodiment, a point different from the first embodiment will be described.

As illustrated in FIG. 18, the light emission device 1I according to the ninth embodiment includes an adhesive layer 70 in addition to the components according to the first embodiment. The adhesive layer 70 has adhesiveness and translucency, and is provided on an entire surface of a surface 40a of a diffusion layer 40, for example. Since the adhesive layer 70 is provided on the diffusion layer 40, stable fixation to a desired position (for example, a lesion portion such as a tumor) in the body is possible.

Furthermore, the adhesive layer 70 has a plurality of through holes 71 respectively connected to through holes 41 of the diffusion layer 40. Each of these through holes 71 is, for example, a mesh portion, but is not limited thereto. Since the adhesive layer 70 has the through holes 71, breathability and liquid permeability of the light emission device 1I are improved.

According to the light emission device 1I in a manner described above, an effect similar to that of the first embodiment can be acquired. Furthermore, since the adhesiveness between the light emission device 1I and the living body is improved, it is possible to improve the therapeutic effect or stability.

### <9-2. Modification Example>

A configuration example of a light emission device 1Ia according to a modification example of the ninth embodiment will be described with reference to FIG. 19. FIG. 19 is a cross-sectional view illustrating the configuration example of the light emission device 1Ia according to the modification example of the ninth embodiment. In the modification example of the ninth embodiment, a point different from the ninth embodiment will be described.

As illustrated in FIG. 19, the light emission device 1Ia according to the modification example of the ninth embodiment has the components according to the ninth embodiment, and an adhesive layer 70 is provided, for example, in an outer peripheral region of a surface 30a of a flexible light guide plate 30 avoiding a diffusion layer 40 on the flexible light guide plate 30. The adhesive layer 70 is formed in, for example, a square ring shape in plan view. Since the adhesive layer 70 is provided on the flexible light guide plate 30, the light emission device 1Ia can be stably fixed to a desired position (for example, a lesion portion such as a tumor) in a body. In addition, by providing the adhesive layer 70 on the surface 30a of the flexible light guide plate 30 while avoiding the diffusion layer 40, it is possible to prevent light emitted from the diffusion layer 40 from being disturbed by the adhesive layer 70.

Note that in some cases, when the light emission device 1Ia is rolled into a scroll shape, unfolding may become difficult due to the presence of the adhesive layer 70. Thus, as in the third embodiment, a non-adhesive layer 60 may be provided on a surface 30b of the flexible light guide plate 30.

According to the light emission device 1Ia in a manner described above, an effect similar to those of the first embodiment and the ninth embodiment can be acquired. Furthermore, as compared with the ninth embodiment, it becomes possible to prevent the light emitted from the diffusion layer 40 from being disturbed by the adhesive layer 70. Thus, light emission efficiency can be improved.

### 10. Tenth Embodiment

### <10-1. Configuration Example of a Light Emission Device>

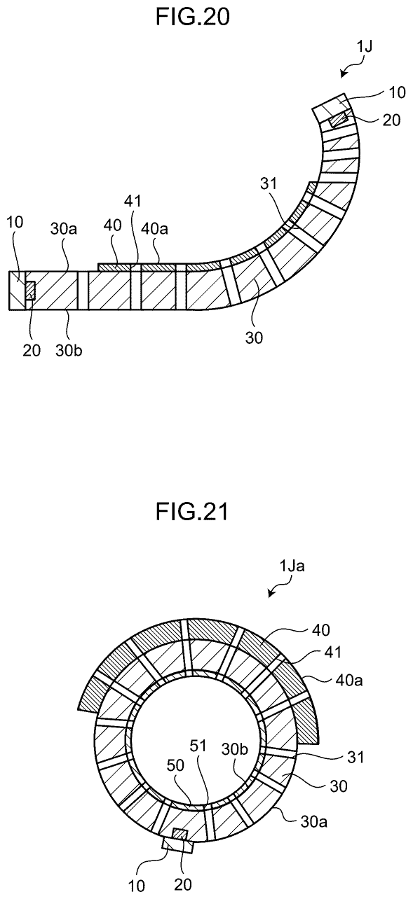

A configuration example of a light emission device 1J according to the tenth embodiment will be described with reference to FIG. 20. FIG. 20 is a cross-sectional view illustrating the configuration example of the light emission device 1J according to the tenth embodiment. In the tenth embodiment, a point different from the first embodiment will be

As illustrated in FIG. 20, the light emission device 1J according to the tenth embodiment includes a new substrate 10 and a plurality of light emitting units 20 in addition to the components according to the first embodiment. That is, the substrates 10 having the light emitting units 20 are respectively provided at both end portions of a flexible light guide plate 30. Since the light enters from both end portions of the flexible light guide plate 30, light emission efficiency is improved and light emission variation is reduced. Note that since a power supply may be complicated, the power supply 12 according to the sixth embodiment may be applied to the tenth embodiment.

According to the light emission device 1J in a manner described above, an effect similar to that of the first embodiment can be acquired. Furthermore, since light is supplied from both end portions of the flexible light guide plate 30 to the flexible light guide plate 30, improvement of light emission efficiency and reduction of light emission variation can be realized.

### <10-2. Modification Example>

A configuration example of a light emission device 1Ja according to a modification example of the tenth embodiment will be described with reference to FIG. 21. FIG. 21 is a cross-sectional view illustrating the configuration example of the light emission device 1Ja according to the modification example of the tenth embodiment. In the modification example of the tenth embodiment, a point different from the modification example of the first embodiment will be described.

As illustrated in FIG. 21, the light emission device 1Ja according to the modification example of the tenth embodiment has the components according to the modification example of the first embodiment, and a flexible light guide plate 30 is formed in, for example, a cylindrical shape. A substrate 10 is provided on an outer peripheral surface of the cylindrical flexible light guide plate 30, and each of light emitting units 20 on the substrate 10 is arranged in such a manner as to emit light toward a center of the cylindrical light emission device 1Ja. A diffusion layer 40 is provided in a desired region on the outer peripheral surface of the flexible light guide plate 30 in such a manner as to face each of the light emitting units 20 on the substrate 10. The reflection layer 50 is provided in an entire region of an inner peripheral surface of the flexible light guide plate 30. Light emission variation is reduced by emission of light to the cylindrical flexible light guide plate 30.

Note that the reflection layer 50 may not be provided in the entire region of the inner peripheral surface of the cylindrical flexible light guide plate 30. However, in order to improve the light emission efficiency, it is preferable that the reflection layer 50 is provided in a region facing the light emitting units 20 on the inner peripheral surface of the flexible light guide plate 30. In addition, since it may be difficult to unfold the light emission device 1Ja in a body, the light emission device 1Ja may have a stretchable structure. Alternatively, the flexible light guide plate 30 may be formed into the cylindrical shape by unfolding of the scroll-shaped flexible light guide plate 30 in the body and joining of both end portions of the flexible light guide plate 30 in the body.

According to the light emission device 1Ja in a manner described above, an effect similar to that of the first embodiment can be acquired. Furthermore, the light emission variation can be reduced by emission of light to the cylindrical flexible light guide plate 30.

### 11. Eleventh Embodiment

### <11-1. Configuration Example of a Light Emission Device>

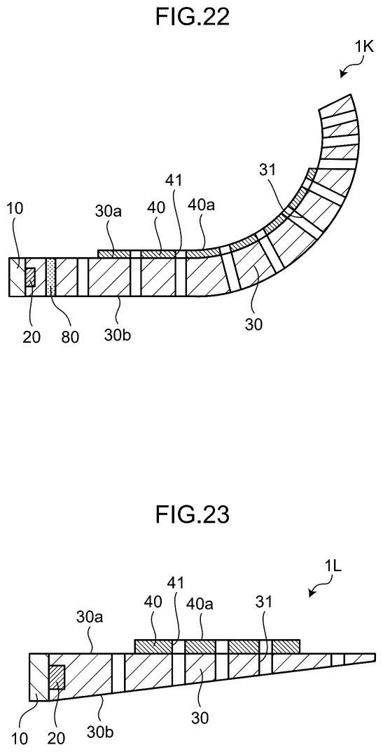

A configuration example of a light emission device 1K according to the eleventh embodiment will be described with reference to FIG. 22. FIG. 22 is a cross-sectional view illustrating the configuration example of the light emission device 1K according to the eleventh embodiment. In the eleventh embodiment, a point different from the first embodiment will be described.

As illustrated in FIG. 22, the light emission device 1K according to the eleventh embodiment includes a release layer 80 in addition to the components according to the first embodiment. The peeling layer 80 has releasability and translucency, and is provided on a part of a flexible light guide plate 30. For example, the release layer 80 is provided between a substrate 10 and a diffusion layer 40 in plan view. As a result, the substrate 10 including each of light emitting units 20 and the flexible light guide plate 30 can be easily separated.

According to the light emission device 1K in a manner described above, an effect similar to that of the first embodiment can be acquired. Furthermore, by providing the release layer 80 on the part of the flexible light guide plate 30, the substrate 10 having the light emitting units 20 can be easily separated from the flexible light guide plate 30.

### 12. Twelfth Embodiment

### <12-1. Configuration Example of a Light Emission Device>

A configuration example of a light emission device 1L according to the twelfth embodiment will be described with reference to FIG. 23. FIG. 23 is a cross-sectional view illustrating the configuration example of the light emission device 1L according to the twelfth embodiment. In the twelfth embodiment, a point different from the first embodiment will be described.

As illustrated in FIG. 23, the light emission device 1L according to the twelfth embodiment includes the components according to the first embodiment, and a thickness of a flexible light guide plate 30 changes according to a separation distance of the flexible light guide plate 30 from a substrate 10. For example, the thickness of the flexible light guide plate 30 decreases as the distance from the substrate 10 increases in the flexible light guide plate 30. A surface 30b of the flexible light guide plate 30 is an inclined surface. In such a manner, it is possible to make a light emission distribution uniform by adjusting a film thickness distribution of the flexible light guide plate 30.

According to the light emission device 1L in a manner described above, an effect similar to that of the first embodiment can be acquired. Furthermore, it is possible to make the light emission distribution uniform by adjusting the film thickness distribution of the flexible light guide plate 30.

### <12-2. First Modification Example>

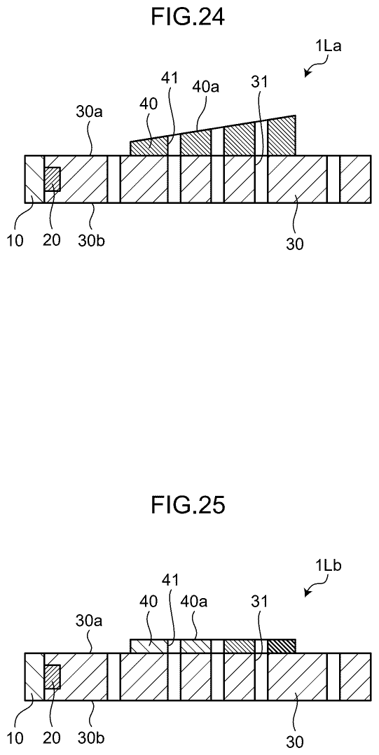

A configuration example of a light emission device 1La according to the first modification example of the twelfth embodiment will be described with reference to FIG. 24. FIG. 24 is a cross-sectional view illustrating the configuration example of the light emission device 1La according to the first modification example of the twelfth embodiment. In the first modification example of the twelfth embodiment, a point different from the first embodiment will be described.

As illustrated in FIG. 24, the light emission device 1La according to the first modification example of the twelfth embodiment includes the components according to the first embodiment, and a thickness of a diffusion layer 40 changes according to a separation distance of the diffusion layer 40 from a substrate 10. For example, the thickness of the diffusion layer 40 increases as the distance from the substrate 10 increases in the diffusion layer 40. A surface 40a of the diffusion layer 40 is an inclined surface. In such a manner, it is possible to make a light emission distribution uniform by adjusting a film thickness distribution of the diffusion layer 40.

According to the light emission device 1La in a manner described above, an effect similar to that of the first embodiment can be acquired. Furthermore, it is possible to make the light emission distribution uniform by adjusting the film thickness distribution of the diffusion layer 40.

### <12-3. Second Modification Example>

A configuration example of a light emission device 1Lb according to the second modification example of the twelfth embodiment will be described with reference to FIG. 25. FIG. 25 is a cross-sectional view illustrating the configuration example of the light emission device 1Lb according to the second modification example of the twelfth embodiment. In the second modification example of the twelfth embodiment, a point different from the first embodiment will be described.

As illustrated in FIG. 25, the light emission device 1Lb according to the second modification example of the twelfth embodiment has the components according to the first embodiment, and a degree of light diffusion of a diffusion layer 40, such as a scattering property of the diffusion layer 40 changes according to a separation distance of the diffusion layer 40 from a substrate 10. For example, the scattering property of the diffusion layer 40 increases as the distance from the substrate 10 increases in the diffusion layer 40. In a case where nanoparticles are mixed in the diffusion layer 40, a refractive index or density of the nanoparticles increases as the distance increases.

According to the light emission device 1Lb in a manner described above, an effect similar to that of the first embodiment can be acquired. Furthermore, it is possible to make a light emission distribution uniform by adjusting a diffusion distribution (such as a scattering distribution) of the diffusion layer 40.

### <12-4. Third Modification Example>

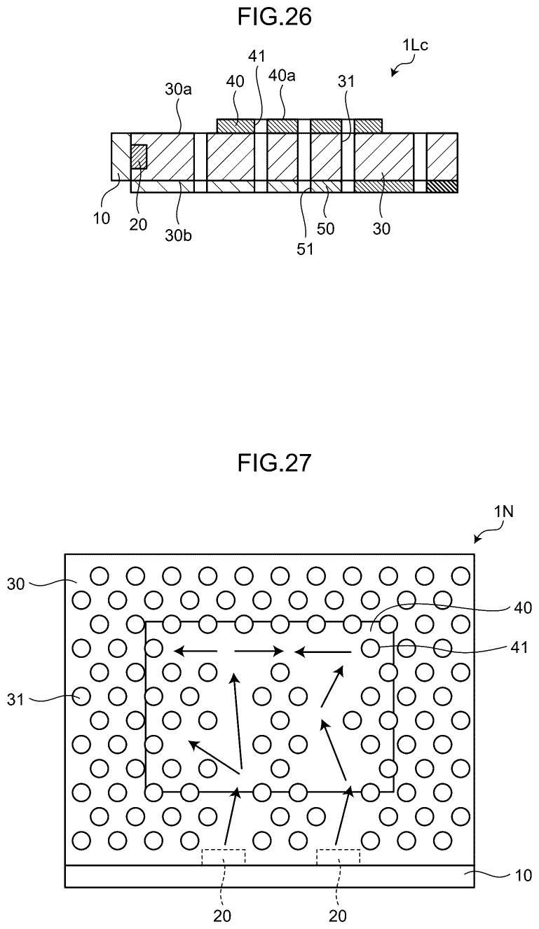

A configuration example of a light emission device 1Lc according to the third modification example of the twelfth embodiment will be described with reference to FIG. 26. FIG. 26 is a cross-sectional view illustrating the configuration example of the light emission device 1Lc according to the third modification example of the twelfth embodiment. In the third modification example of the twelfth embodiment, a point different from the first embodiment will be described.

As illustrated in FIG. 26, the light emission device 1Lc according to the third modification example of the twelfth embodiment includes a reflection layer 50 in addition to the components according to the first embodiment. The reflection layer 50 is formed in an entire region of a surface 30b of a flexible light guide plate 30, and a degree of light reflection of the reflection layer 50, such as a scattering property of the reflection layer 50 changes according to a separation distance of the reflection layer 50 from a substrate 10. For example, the scattering property of the reflection layer 50 increases as the distance from the substrate 10 increases in the reflection layer 50.

According to the light emission device 1Lc in a manner described above, an effect similar to that of the first embodiment can be acquired. Furthermore, it is possible to make a light emission distribution uniform by adjusting a reflection distribution (such as a scattering distribution) of the reflection layer 50.

Note that in order to make the light emission distribution uniform by adjusting the film thickness distribution, the diffusion distribution, and the reflection distribution, any two or more of the twelfth embodiment and the first to third modification examples of the twelfth embodiment may be appropriately combined. In addition, in a case where the reflection layer 50 is used, the diffusion layer 40 may not be included.

### 13. Thirteenth Embodiment

### <13-1. Configuration Example of a Light Emission Device>

A configuration example of a light emission device 1N according to the thirteenth embodiment will be described with reference to FIG. 27. FIG. 27 is a plan view illustrating the configuration example of the light emission device 1N according to the thirteenth embodiment. In the thirteenth embodiment, a point different from the first embodiment will be described.

As illustrated in FIG. 27, the light emission device 1N according to the thirteenth embodiment has the components according to the first embodiment, and through holes 31 of a flexible light guide plate 30 configure a photonic crystal. Since the photonic crystal is formed in the flexible light guide plate 30, light emitted from light emitting units 20 can be confined, and light utilization efficiency can be increased. The photonic crystal uses a refraction difference between the flexible light guide plate 30 and air.

According to the light emission device 1N in a manner described above, an effect similar to that of the first embodiment can be acquired. Furthermore, by applying the photonic crystal to the flexible light guide plate 30, it is possible to improve the light emission efficiency and reduce the loss of light.

### 14. Fourteenth Embodiment>

### <14-1. Configuration Example of a Light Emission Device>

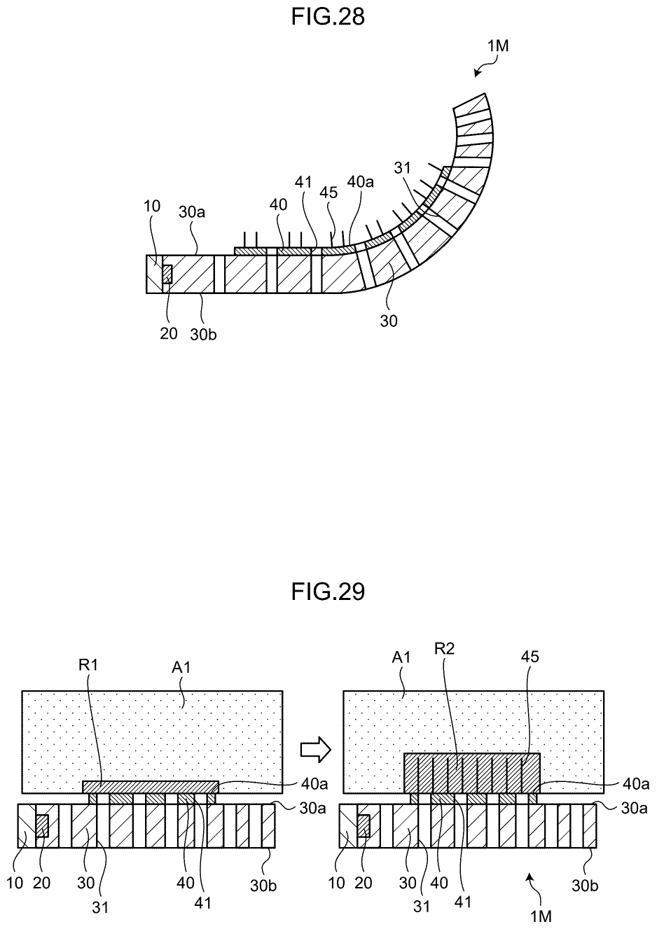

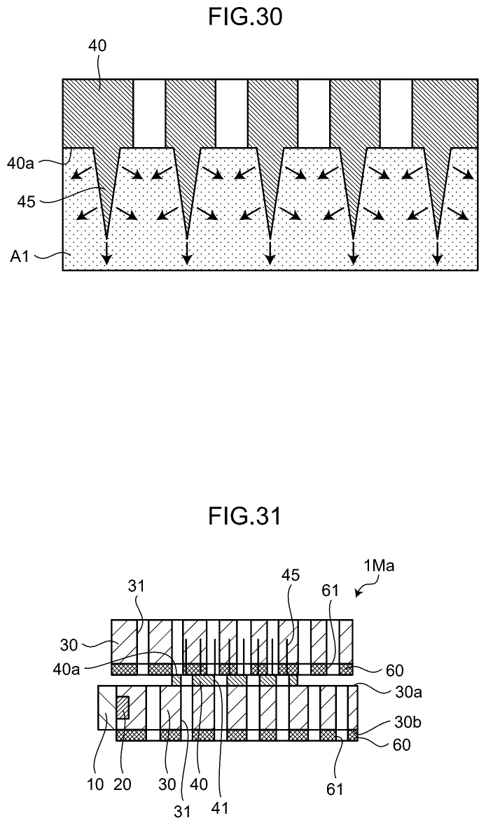

A configuration example of a light emission device 1M according to the fourteenth embodiment will be described with reference to FIG. 28 to FIG. 30. FIG. 28 is a cross-sectional view illustrating the configuration example of the light emission device 1M according to the fourteenth embodiment. FIG. 29 is a view for describing a light reaching range based on presence or absence of a protrusion portion 45 according to the fourteenth embodiment. FIG. 30 is a view illustrating the protrusion portion 45 according to the fourteenth embodiment. In the fourteenth embodiment, a point different from the first embodiment will be described.

As illustrated in FIG. 28, the light emission device 1M according to the fourteenth embodiment includes the components according to the first embodiment, and a diffusion layer 40 includes a plurality of the protrusion portions 45. These protrusion portions 45 are formed in a needle shape, and are provided in a matrix shape in an entire region of a surface 40a of the diffusion layer 40, for example. Although not needing to be specifically aligned, the protrusion portions 45 are preferably arranged in a matrix. Rows and columns may be aligned at the same pitch or may be aligned randomly. In addition, each of the protrusion portions 45 may be provided not in the entire region of the surface 40a of the diffusion layer 40 but in a partial region of the surface 40a of the diffusion layer 40.

Here, as illustrated in FIG. 29, in a case where the protrusion portions 45 are not present, a region R1 is a light reaching range. In this case, a light reaching depth is shallow. On the other hand, in a case where the protrusion portions 45 are present, a region R2 is a light reaching range. As illustrated in FIG. 30, the presence of the protrusion portions 45 improves the light reaching depth. Each of the protrusion portions 45 is stuck into a living body A1 (for example, a lesion such as a tumor) and guides the light into the living body A1. As a result, the light reaching range is widened, and the light reaching depth is improved.

Each of the protrusion portions 45 is formed of, for example, PDMS, or PDMS and nanoparticles. In addition, each of the protrusion portions 45 may be formed of, for example, Si or the like. A shape of the protrusion portions 45 is, for example, a needle shape or a pyramid shape. A size of the protrusion portions 45 is, for example, a micro size or a nano size in order to control an influence on the living body, and a thickness of the protrusion portions 45 is preferably thin, but a certain degree of hardness is required.

According to the light emission device 1M in a manner described above, an effect similar to that of the first embodiment can be acquired. Furthermore, the presence of each of the protrusion portions 45 can improve the light reaching depth. Note that in a case where the diffusion layer 40 is not included in a flexible light guide plate 30, each of the protrusion portions 45 may be provided on a surface 30a of the flexible light guide plate 30.

### <14-2. First Modification Example>

A configuration example of a light emission device 1Ma according to a first modification example of the fourteenth embodiment will be described with reference to FIG. 31. FIG. 31 is a cross-sectional view illustrating the configuration example of the light emission device 1Ma according to the first modification example of the fourteenth embodiment. In the fourteenth embodiment, a point different from the third embodiment will be described.

As illustrated in FIG. 31, the light emission device 1Ma according to the first modification example of the fourteenth embodiment includes the components according to the third embodiment, and a diffusion layer 40 includes a plurality of protrusion portions 45. These protrusion portions 45 are similar to those in the fourteenth embodiment.

Since a non-adhesive layer 60 is provided on a surface 30b of a flexible light guide plate 30, portions of the flexible light guide plate 30 (front and back of the flexible light guide plate 30) are easily separated even when adhering to each other. In addition, since the flexible light guide plate 30 is, for example, a meshed film having through holes 31, even when the protrusion portions 45 are stuck into the flexible light guide plate 30, damage of the flexible light guide plate 30 and the protrusion portions 45 is less likely to be generated, and adhesion or the like between the portions of the flexible light guide plate 30 due to the protrusion portions 45 is less likely to be generated.

According to the light emission device 1Ma in a manner described above, an effect similar to that of the third embodiment can be acquired. Furthermore, the presence of each of the protrusion portions 45 can improve the light reaching depth.

### <14-3. Second Modification Example>

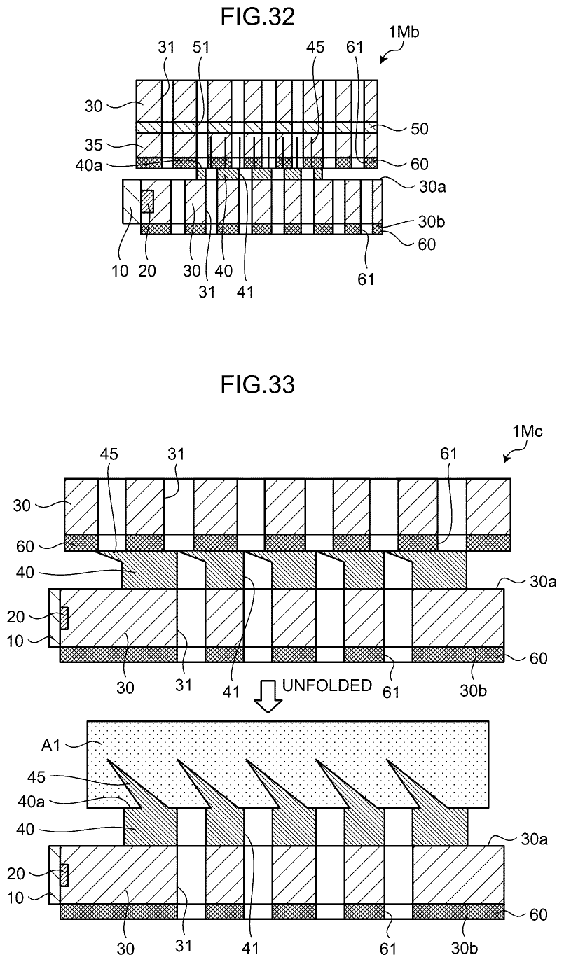

A configuration example of a light emission device 1Mb according to the second modification example of the fourteenth embodiment will be described with reference to FIG. 32. FIG. 32 is a cross-sectional view illustrating the configuration example of the light emission device 1Mb according to the second modification example of the fourteenth embodiment. In the second modification example of the fourteenth embodiment, a point different from the modification example of the third embodiment will be described.

As illustrated in FIG. 32, the light emission device 1Mb according to the first modification example of the fourteenth embodiment includes a protrusion absorption layer 35 in addition to the components according to the modification example of the third embodiment, and a diffusion layer 40 includes a plurality of protrusion portions 45. These protrusion portions 45 are similar to those in the fourteenth embodiment. The protrusion absorption layer 35 is a layer which is provided between a reflection layer 50 and a non-adhesive layer 60, and into which each of the protrusion portions 45 is stuck. A thickness of the protrusion absorption layer 35 is set in such a manner that the protrusion portions 45 that are stuck into the protrusion absorption layer 35 do not reach the reflection layer 50. The protrusion absorption layer 35 is formed of, for example, PDMS or the like.

For example, in a case where the reflection layer 50 is formed of metal or the like, the protrusion portions 45 may be damaged when the protrusion portions 45 come into contact with the reflection layer 50. Thus, damage to the protrusion portions 45 can be controlled by provision of the protrusion absorption layer 35 between the reflection layer 50 and the non-adhesive layer 60.

According to the light emission device 1Mb in a manner described above, it is possible to acquire an effect similar to that of the modification example of the third embodiment. Furthermore, the presence of each of the protrusion portions 45 can improve the light reaching depth. In addition, damage to the protrusion portions 45 can be controlled by provision of the protrusion absorption layer 35.

### <14-4. Third Modification Example>

A configuration example of a light emission device 1Mc according to the third modification example of the fourteenth embodiment will be described with reference to FIG. 33. FIG. 33 is a cross-sectional view illustrating the configuration example of the light emission device 1Mc according to the third modification example of the fourteenth embodiment. In the fourteenth embodiment, a point different from the first modification example of the fourteenth embodiment will be described.

As illustrated in FIG. 33, the light emission device 1Mc according to the third modification example of the fourteenth embodiment has the components according to the first modification example of the fourteenth embodiment, and each of protrusion portions 45 is similar to that of the fourteenth embodiment but is formed to extend obliquely with respect to a surface 30a of a flexible light guide plate 30.

When each of the protrusion portions 45 is formed obliquely, each of the protrusion portions 45 does not interfere in the scroll-shaped light emission device 1Mc, and each of the protrusion portions 45 is stuck into a living body A1 (for example, a lesion such as a tumor) when the scroll-shaped light emission device 1Mc is unfolded and attached to a desired position. Note that in the light emission device 1Mc in an unfolded state, each of the protrusion portions 45 is inclined with respect to the surface 30a of the flexible light guide plate 30.

According to the light emission device 1Mc in a manner described above, it is possible to acquire an effect similar to that of the first modification example of the fourteenth embodiment. Furthermore, by making each of the protrusion portions 45 oblique, it is possible to control damage to the protrusion portions 45 of a case where the light emission device 1Mc is rolled into the scroll shape.

### 15. Fifteenth Embodiment

### <15-1. Configuration Example of a Light Emission Device>

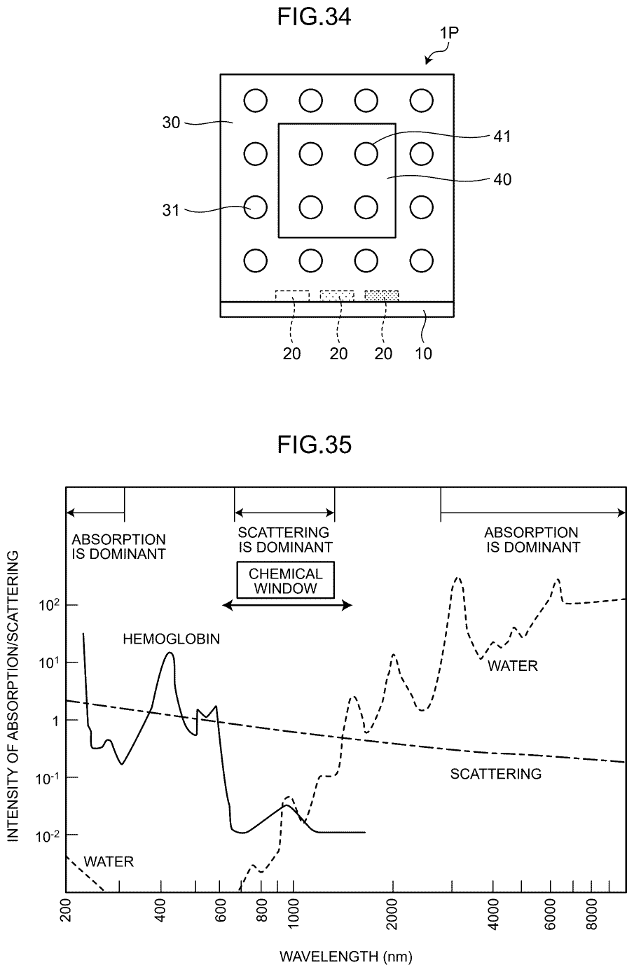

A configuration example of a light emission device 1P according to the fifteenth embodiment will be described with reference to FIG. 34 and FIG. 35. FIG. 34 is a plan view illustrating the configuration example of the light emission device 1P according to the fifteenth embodiment. FIG. 35 is a graph illustrating a relationship between intensity of light absorption/scattering and a wavelength according to the fifteenth embodiment. In the fifteenth embodiment, a point different from the first embodiment will be described.

As illustrated in FIG. 34, the light emission device 1P according to the fifteenth embodiment includes the components according to the first embodiment, and light emitting units 20 respectively emit pieces of light having different wavelengths.

As illustrated in FIG. 35, biological permeability (tissue permeability) is affected by absorption of hemoglobin and water. The biological permeability has a relationship of near-infrared (NIR) to red>green>blue. By using a plurality of wavelengths, it is possible to control a biological depth and an action by light.

According to the light emission device 1P in a manner described above, an effect similar to that of the first embodiment can be acquired. Furthermore, it is possible to change the biological depth and the action by the light depending on the wavelength. For example, in a case of PDT, it is possible to use violet light, and it is possible to improve a cancer therapeutic effect on a surface.

### <15-2. Modification Example>

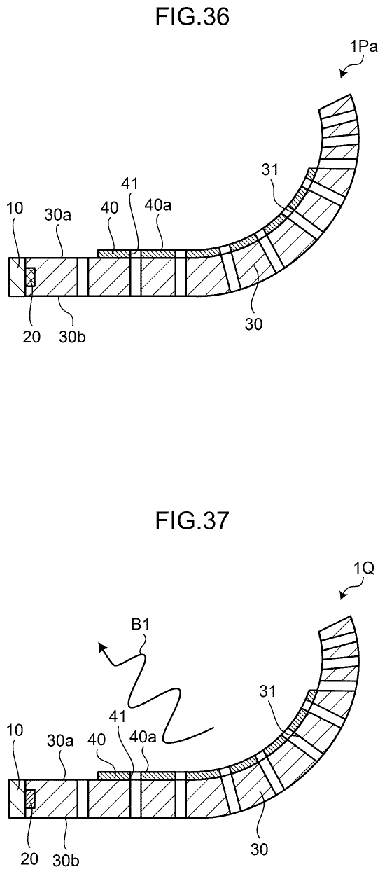

A configuration example of a light emission device 1Pa according to the modification example of the fifteenth embodiment will be described with reference to FIG. 36. FIG. 36 is a cross-sectional view illustrating the configuration example of the light emission device 1Pa according to the modification example of the fifteenth embodiment. In the fifteenth embodiment, a point different from the first embodiment will be described.

As illustrated in FIG. 36, the light emission device 1Pa according to the modification example of the fifteenth embodiment includes the components according to the first embodiment, and a flexible light guide plate 30 or a diffusion layer 40 has a color conversion function of converting color of light emitted by a light emitting unit 20. The flexible light guide plate 30 or the diffusion layer 40 includes, for example, a color conversion phosphor in order to realize the color conversion function.

As the color conversion phosphor, for example, a color conversion quantum dot (QD) is used. The color conversion QD also has a diffusion effect, and is excited by light of ultraviolet (UV), violet, blue, or the like. In the PDT, violet light is used. As the color conversion QD, for example, CdSe, InP, ZnSe, ZnSeTe, CsPbI3, PbS, or the like is used. Note that a photosensitizer may have an up-conversion function.

According to the light emission device 1Pa in a manner described above, an effect similar to those of the first embodiment and the fifteenth embodiment can be acquired. For example, it is possible to change a biological depth and an action by light depending on a wavelength.

### 16. Sixteenth Embodiment

### <16-1. Configuration Example of a Light Emission Device>

A configuration example of a light emission device 1Q according to the sixteenth embodiment will be described with reference to FIG. 37. FIG. 37 is a cross-sectional view illustrating the configuration example of the light emission device 1Q according to the sixteenth embodiment. In the thirteenth embodiment, a point different from the first embodiment will be described.

As illustrated in FIG. 37, the light emission device 1Q according to the sixteenth embodiment includes the components according to the first embodiment, and a flexible light guide plate 30 has a function of emitting one or both of oxygen and a photosensitizer. In the example of FIG. 37, an arrow B1 indicates an emission of one or both of oxygen and the photosensitizer.

For example, oxygen is stored in holes or a metal-organic framework (MOF) of the flexible light guide plate 30, and the stored oxygen is emitted to supply oxygen into a body. The holes correspond to through holes 31 and holes in the flexible light guide plate 30. Usually, although a deep organ has less oxygen, there is also a therapeutic effect on the deep organ having less oxygen. Thus, the therapeutic effect of PDT can be improved.

In addition, for example, a photosensitizer is stored in the holes, the MOF, or the like of the flexible light guide plate 30, and the stored photosensitizer is emitted to supply the photosensitizer into the body. As a result, it becomes unnecessary to administer the photosensitizer from the outside.

Note that a photosynthesis layer may be added to the light emission device 1Q and generate oxygen with water and light. However, in a case where the photosynthesis layer is used, light utilization efficiency may be reduced. Thus, as described above, it is preferable to use the holes or the MOF of the flexible light guide plate 30.

According to the light emission device 10 in a manner described above, an effect similar to that of the first embodiment can be acquired. Furthermore, since oxygen can be emitted, improvement of the therapeutic effect of PDT can be realized. In addition, since it becomes possible to emit the photosensitizer, administration of the photosensitizer becomes unnecessary.

### <17. Action and Effect According to Each Embodiment>

As described above, according to each of the embodiments, the light emission device (such as the light emission device 1A to 10, 1Aa, 1Ba, 1Ca, 1Fa, 1Ia, 1Ja, 1La to 1Lc, 1Ma to 1Mc, or 1Pa) includes the substrate 10, the light emitting units 20 that are provided on the substrate 10 and emit light, and the flexible light guide plate 30 that is joined to the substrate 10, guides the light emitted by the light emitting units 20, and has the plurality of through holes 31. As a result, breathability and liquid permeability of the light emission device are improved, and biocompatibility can be improved. In addition, since the number of the light emitting units 20 can be reduced as compared with a case where a large number of light emitting units 20 are provided on the entire surface of the light emission device as in the related art, improvement of flexibility and reduction in a cost can be realized.

Furthermore, the substrate 10 may be provided at the end portion of the flexible light guide plate 30 (see FIG. 2 and the like). As a result, improvement of the flexibility can be reliably realized.

Furthermore, the flexible light guide plate 30 may be joined to the substrate 10 in such a manner as to cover the light emitting units 20 on the substrate 10 (see FIG. 2 and the like). This makes it possible to protect the light emitting units 20 from gas, liquid, and the like.

In addition, the light emission device 1A may further include a diffusion layer 40 that is provided on the surface 30a of the flexible light guide plate 30, that diffuses the light guided by the flexible light guide plate 30, and that has a plurality of through holes 41 respectively connected to the plurality of through holes 31 of the flexible light guide plate 30 (see FIG. 2). This makes it possible to adjust the light emitting region while improving the biocompatibility.

In addition, the light emission device 1Aa may further include a reflection layer 50 provided on the surface 30b of the flexible light guide plate 30, reflecting the light guided by the flexible light guide plate 30, and having a plurality of through holes 51 respectively connected to the plurality of through holes 31 of the flexible light guide plate 30 (see FIG. 3). This makes it possible to adjust the light emitting region while improving the biocompatibility.

In addition, the light emission device 1B may further include a reflection layer 50A provided on an outer peripheral side of the surface 30a of the flexible light guide plate 30, reflecting the light guided by the flexible light guide plate 30, and having a plurality of through holes 51 respectively connected to the plurality of through holes 31 of the flexible light guide plate 30, and a reflection layer 50B provided on the surface 30b of the flexible light guide plate 30, reflecting the light guided by the flexible light guide plate 30, and having a plurality of through holes 51 respectively connected to the plurality of through holes 31 of the flexible light guide plate 30 (see FIG. 7). This makes it possible to adjust the light emitting region while improving the biocompatibility.

In addition, the light emission device 1Ba further includes a diffusion layer 40 provided on the surface 30a of the flexible light guide plate 30, diffusing the light guided by the flexible light guide plate 30, and having a plurality of through holes 41 respectively connected to the plurality of through holes 31 of the flexible light guide plate 30, and a reflection layer 50B provided on the surface 30b of the flexible light guide plate 30, reflecting the light guided by the flexible light guide plate 30, and having a plurality of through holes 51 respectively connected to the plurality of through holes 31 of the flexible light guide plate 30 (see FIG. 8). This makes it possible to adjust the light emitting region while improving the biocompatibility.

In addition, the light emission device 1C may further include a non-adhesive layer 60 provided on the surface 30b of the flexible light guide plate 30 and having a plurality of through holes 61 respectively connected to the plurality of through holes 31 of the flexible light guide plate 30 (see FIG. 9). As a result, even in a case where the light emission device 1C is rolled into a scroll shape, it is possible to control adhesion between the portions of the flexible light guide plate 30. Thus, it is possible to facilitate unfolding of the scroll-shaped light emission device 1C.

In addition, the light emission device 1Ca may further include a reflection layer 50 that is provided on the surface 30b of the flexible light guide plate 30, reflects the light guided by the flexible light guide plate 30, and has a plurality of through holes 51 respectively connected to the plurality of through holes 31 of the flexible light guide plate 30, and a non-adhesive layer 60 that is provided on a surface on an opposite side of a living body attachment side of the reflection layer 50, and has a plurality of through holes 61 respectively connected to the plurality of through holes 31 of the flexible light guide plate 30 (see FIG. 10). As a result, even in a case where the light emission device 1Ca is rolled into the scroll shape, it is possible to control adhesion between the portions of the flexible light guide plate 30. Thus, it is possible to facilitate unfolding of the scroll-shaped light emission device 1Ca.

Furthermore, the flexible light guide plate 30 may have a function of diffusing the light emitted by the light emitting units 20 (see FIG. 11). As a result, the configuration can be simplified as compared with a case where the diffusion layer 40 is provided.

Furthermore, the substrate 10 may be positioned at an end portion of the flexible light guide plate 30, and the light emitting units 20 may be provided to emit light toward the surface 30a of the flexible light guide plate 30 (see FIG. 12). As a result, since the thickness of the flexible light guide plate 30 can be controlled, the flexibility can be improved.

Furthermore, the light emission device 1F or the light emission device 1Fa may further include a power supply 12 or a wireless power feeding unit 14 that is provided on the substrate 10 and supplies power to the light emitting units 20 (see FIG. 13 to FIG. 15, and the like). As a result, since the light emission device 1F includes the power supply 12 and the light emission device 1Fa includes the wireless power feeding unit 14, it becomes possible to drive the light emission device 1F or the light emission device 1Fa in a stand-alone manner.

Furthermore, the light emission device 1F or the light emission device 1Fa may further include a circuit 13 that is provided on the substrate 10 and that is for communication or control (see FIG. 13 to FIG. 15, and the like). As a result, in a case where communication or control (such as current control) is necessary, since the light emission device 1F or the light emission device 1Fa includes the circuit 13 for communication or control, it is possible to deal with necessary communication or control.

Furthermore, the light emission device 1G may further include a sensor 15 that is provided on the substrate 10 and that detects a device state or a biological state (see FIG. 16). As a result, since the device state or the biological state can be detected and fed back to the driving condition of the light emitting unit 20 or the like, improvement of the safety or the therapeutic effect can be realized.

In addition, the light emission device 1H may further include a stent S1 around which the flexible light guide plate 30 is wound (see FIG. 17). As a result, since the light emission device 1H is used in combination with the stent S1, the light emission device 1H can be fixed and used in the ductal system such as the bile duct.

In addition, the light emission device 1I may further include a diffusion layer 40 provided on the surface 30a of the flexible light guide plate 30, diffusing the light guided by the flexible light guide plate 30, and having a plurality of through holes 41 respectively connected to the plurality of through holes 31 of the flexible light guide plate 30, and an adhesive layer 70 provided on the surface 40a of the diffusion layer 40, and having a plurality of through holes 71 respectively connected to the plurality of through holes 31 of the flexible light guide plate 30 (see FIG. 18). As a result, since the adhesiveness between the light emission device 1I and the living body is improved, it is possible to improve the therapeutic effect or stability.

In addition, the light emission device 1Ia may further include an adhesive layer 70 provided on the surface 30a of the flexible light guide plate 30 and having a plurality of through holes 71 respectively connected to the plurality of through holes 31 of the flexible light guide plate 30 (see FIG. 19). As a result, it becomes possible to prevent the light emitted from the diffusion layer 40 from being disturbed by the adhesive layer 70. Thus, the light emission efficiency can be improved.

In addition, a plurality of the substrates 10 and a plurality of the light emitting units 20 may be provided, and the plurality of substrates 10 may have light emitting units 20 and be provided at both end portions of the flexible light guide plate 30, respectively (see FIG. 20). As a result, since the light is supplied from the both end portions of the flexible light guide plate 30 to the flexible light guide plate 30, improvement of the light emission efficiency and reduction of the light emission variation can be realized.

In addition, the flexible light guide plate 30 may be formed in a cylindrical shape (see FIG. 21). As a result, the light emission variation can be reduced by emission of the light to the cylindrical flexible light guide plate 30.

In addition, the substrate 10 may be provided at the end portion of the flexible light guide plate 30, and the light emission device 1K may further include a release layer 80 provided on a side of the substrate 10 of the flexible light guide plate 30 (see FIG. 22). As a result, the substrate 10 having the light emitting units 20 can be easily separated from the flexible light guide plate 30.

Furthermore, the substrate 10 is provided at the end portion of the flexible light guide plate 30, and the thickness of the flexible light guide plate 30 changes according to the Separation distance of the flexible light guide plate 30 from the substrate 10 (see FIG. 23). As a result, the film thickness distribution of the flexible light guide plate 30 can be adjusted, and a light emission distribution can be made uniform.

In addition, the light emission device 1La may further include a diffusion layer 40 provided on the surface 30a of the flexible light guide plate 30, diffusing the light guided by the flexible light guide plate 30, and having a plurality of through holes 41 respectively connected to the plurality of through holes 31 of the flexible light guide plate 30, the substrate 10 may be provided at the end portion of the flexible light guide plate 30, and the thickness of the diffusion layer 40 may change according to the separation distance of the diffusion layer 40 from the substrate 10 (see FIG. 24). As a result, the film thickness distribution of the diffusion layer 40 can be adjusted, and the light emission distribution can be made uniform.

In addition, the light emission device 1Lb may further include a diffusion layer 40 provided on the surface 30a of the flexible light guide plate 30, diffusing the light guided by the flexible light guide plate 30, and having a plurality of through holes 41 respectively connected to the plurality of through holes 31 of the flexible light guide plate 30, the substrate 10 may be provided at the end portion of the flexible light guide plate 30, and the degree of the light diffusion of the diffusion layer 40 may change according to the separation distance of the diffusion layer 40 from the substrate 10 (see FIG. 25). Thus, the diffusion distribution of the diffusion layer 40 can be adjusted, and the light emission distribution can be made uniform.

Furthermore, the light emission device 1Lc may further include a reflection layer 50 provided on the surface 30b of the flexible light guide plate 30, reflecting the light guided by the flexible light guide plate 30, and having a plurality of through holes 51 respectively connected to the plurality of through holes 31 of the flexible light guide plate 30, the substrate 10 may be provided at the end portion of the flexible light guide plate 30, and the degree of the light reflection of the reflection layer 50 may change according to the separation distance of the reflection layer 50 from the substrate 10 (see FIG. 26). Thus, the reflection distribution of the reflection layer 50 can be adjusted, and the light emission distribution can be made uniform.

Furthermore, the plurality of through holes 31 of the flexible light guide plate 30 may form a photonic crystal (see FIG. 27). Thus, by applying the photonic crystal to the flexible light guide plate 30, it is possible to improve light emission efficiency and reduce a loss of light.

In addition, the light emission device 1M may further include a plurality of protrusion portions 45 formed on the surface 30a of the flexible light guide plate 30 (see FIG. 28 to FIG. 33). As a result, the presence of each of the protrusion portions 45 can improve the light reaching depth.

In addition, the light emission device 1M may further include a diffusion layer 40 provided on the surface 30a of the flexible light guide plate 30, diffusing the light guided by the flexible light guide plate 30, and having a plurality of through holes 41 respectively connected to the plurality of through holes 31 of the flexible light guide plate 30, and a plurality of protrusion portions 45 formed on the surface 40a of the diffusion layer 40 (see FIG. 28 to FIG. 33). As a result, the presence of each of the protrusion portions 45 can improve the light reaching depth.

Furthermore, each of the plurality of protrusion portions 45 may be formed in such a manner as to extend obliquely with respect to the surface 30a of the flexible light guide plate 30 (see FIG. 33). By making each of the protrusion portions 45 oblique, it is possible to control the damage to the protrusion portions 45 of a case where the light emission device 1Mc is rolled into the scroll shape.

Furthermore, a plurality of the light emitting units 20 may be provided, and the plurality of light emitting units 20 may respectively emit pieces of light having different wavelengths (see FIG. 34). As a result, it is possible to change the biological depth and the action by the light depending on the wavelengths.

In addition, the flexible light guide plate 30 may have a color conversion function of converting the color of the light emitted by the light emitting units 20 (see FIG. 36). As a result, it is possible to change the biological depth and the action by the light depending on the wavelengths.

In addition, the light emission device 1Pa may further include a diffusion layer 40 provided on the surface 30a of the flexible light guide plate 30, diffusing the light guided by the flexible light guide plate 30, and having a plurality of through holes 41 respectively connected to the plurality of through holes 31 of the flexible light guide plate 30, and the diffusion layer 40 may have a color conversion function of converting the color of the light emitted by the light emitting unit 20 (see FIG. 36). As a result, it is possible to change the biological depth and the action by the light depending on the wavelengths.

In addition, the flexible light guide plate 30 may have a function of emitting oxygen (see FIG. 37). As a result, since oxygen can be emitted, an improvement of the therapeutic effect of PDT can be realized.

In addition, the flexible light guide plate 30 may have a function of emitting a photosensitizer (see FIG. 37). As a result, since it becomes possible to emit the photosensitizer, administration of the photosensitizer becomes unnecessary.

### 18. Other Embodiments

The above-described embodiments (including modification examples) may be implemented in various different forms or modification examples other than the above-described embodiments. In addition, the configurations, procedures, specific names, and information including various kinds of data, parameters, and the like in the above document or in the drawings can be arbitrarily modified unless otherwise specified. For example, various kinds of information illustrated in each of the drawings are not limited to the illustrated information. Also, the above-described embodiments (including modification examples) can be arbitrarily combined in a range in which the contents do not contradict with each other. In addition, an effect described in the present description is merely an example and is not a limitation, and there may be a different effect.

### 19. Supplementary Note

Note that the present technology can also have the following configurations.

(1)

A living body attachment-type light emission device comprising:
a substrate;
a light emitting unit that is provided on the substrate and emits light; and
a flexible light guide plate that is joined to the substrate, guides the light emitted by the light emitting unit, and has a plurality of through holes.
   (2)

A living body attachment-type light emission device according to (1), wherein
the substrate is provided at an end portion of the flexible light guide plate.
   (3)

A living body attachment-type light emission device according to (1) or (2), wherein
the flexible light guide plate is joined to the substrate in such a manner as to cover the light emitting unit.
   (4)

A living body attachment-type light emission device according to any one of (1) to (3), further comprising
a diffusion layer that is provided on a surface on a living body attachment side of the flexible light guide plate, diffuses the light guided by the flexible light guide plate, and has a plurality of through holes respectively connected to the plurality of through holes of the flexible light guide plate.
   (5)

A living body attachment-type light emission device according to any one of (1) to (4), further comprising
a reflection layer that is provided on a surface on an opposite side of a living body attachment side of the flexible light guide plate, reflects the light guided by the flexible light guide plate, and has a plurality of through holes respectively connected to the plurality of through holes of the flexible light guide plate.
   (6)

A living body attachment-type light emission device according to any one of (1) to (4), further comprising:
a first reflection layer that is provided on an outer peripheral side of a surface on a living body attachment side of the flexible light guide plate, reflects the light guided by the flexible light guide plate, and has a plurality of through holes respectively connected to the plurality of through holes of the flexible light guide plate; and
a second reflection layer that is provided on a surface on an opposite side of the living body attachment side of the flexible light guide plate, reflects the light guided by the flexible light guide plate, and has a plurality of through holes respectively connected to the plurality of through holes of the flexible light guide plate.
   (7)

A living body attachment-type light emission device according to any one of (1) to (5), further comprising:
a diffusion layer that is provided on a surface on a living body attachment side of the flexible light guide plate, diffuses the light guided by the flexible light guide plate, and has a plurality of through holes respectively connected to the plurality of through holes of the flexible light guide plate; and
a reflection layer that is provided on a surface on an opposite side of the living body attachment side of the flexible light guide plate, reflects the light guided by the flexible light guide plate, and has a plurality of through holes respectively connected to the plurality of through holes of the flexible light guide plate.
   (8)

A living body attachment-type light emission device according to any one of (1) to (7), further comprising
a non-adhesive layer that is provided on a surface on an opposite side of a living body attachment side of the flexible light guide plate, and has a plurality of through holes respectively connected to the plurality of through holes of the flexible light guide plate.
   (9)

A living body attachment-type light emission device according to any one of (1) to (5), further comprising:
a reflection layer that is provided on a surface on an opposite side of a living body attachment side of the flexible light guide plate, reflects the light guided by the flexible light guide plate, and has a plurality of through holes respectively connected to the plurality of through holes of the flexible light guide plate; and
a non-adhesive layer that is provided on a surface on the opposite side of the living body attachment side of the reflection layer, and has a plurality of through holes respectively connected to the plurality of through holes of the flexible light guide plate.
   (10)

A living body attachment-type light emission device according to any one of (1) to (9), wherein
the flexible light guide plate has a function of diffusing the light emitted by the light emitting unit.
   (11)

A living body attachment-type light emission device according to any one of (1) to (10), wherein
the substrate is positioned at an end portion of the flexible light guide plate, and the light emitting unit is provided in such a manner as to emit light toward a surface on a living body attachment side of the flexible light guide plate.
   (12)

A living body attachment-type light emission device according to any one of (1) to (11), further comprising
a power supply or a wireless power feeding unit that is provided on the substrate and that supplies power to the light emitting unit.
   (13)

A living body attachment-type light emission device according to any one of (1) to (12), further comprising
a circuit that is provided on the substrate and that is for communication or control.
   (14)

A living body attachment-type light emission device according to any one of (1) to (13), further comprising
a sensor that is provided on the substrate and that detects a device state or a biological state.
   (15)

A living body attachment-type light emission device according to any one of (1) to (14), further comprising
a stent around which the flexible light guide plate is wound.
   (16)

A living body attachment-type light emission device according to any one of (1) to (15), further including:
a diffusion layer that is provided on a surface on a living body attachment side of the flexible light guide plate, diffuses the light guided by the flexible light guide plate, and has a plurality of through holes respectively connected to the plurality of through holes of the flexible light guide plate; and
an adhesive layer that is provided on a surface on the living body attachment side of the diffusion layer, and has a plurality of through holes respectively connected to the plurality of through holes of the flexible light guide plate.
   (17)

A living body attachment-type light emission device according to any one of (1) to (15), further including
an adhesive layer that is provided on a surface on a living body attachment side of the flexible light guide plate, and has a plurality of through holes respectively connected to the plurality of through holes of the flexible light guide plate.
   (18)

A living body attachment-type light emission device according to any one of (1) to (17), in which
a plurality of the substrates and a plurality of the light emitting units are provided, and
the plurality of substrates respectively includes the light emitting units and is respectively provided at both end portions of the flexible light guide plate.
   (19)

A living body attachment-type light emission device according to any one of (1) to (17), in which
the flexible light guide plate is formed in a cylindrical shape.
   (20)

A living body attachment-type light emission device according to any one of (1) to (19), in which
the substrate is provided at an end portion of the flexible light guide plate, and
a release layer provided on a side of the substrate of the flexible light guide plate is further included.
   (21)

A living body attachment-type light emission device according to any one of (1) to (20), in which
the substrate is provided at an end portion of the flexible light guide plate, and
a thickness of the flexible light guide plate changes according to a separation distance of the flexible light guide plate from the substrate.
   (22)

A living body attachment-type light emission device according to any one of (1) to (21), further including
a diffusion layer that is provided on a surface on a living body attachment side of the flexible light guide plate, diffuses the light guided by the flexible light guide plate, and has a plurality of through holes respectively connected to the plurality of through holes of the flexible light guide plate, in which
the substrate is provided at an end portion of the flexible light guide plate, and
a thickness of the diffusion layer changes according to a separation distance of the diffusion layer from the substrate.
   (23)

A living body attachment-type light emission device according to any one of (1) to (22), further including
a diffusion layer that is provided on a surface on a living body attachment side of the flexible light guide plate, diffuses the light guided by the flexible light guide plate, and has a plurality of through holes respectively connected to the plurality of through holes of the flexible light guide plate, in which
the substrate is provided at an end portion of the flexible light guide plate, and
a degree of light diffusion of the diffusion layer changes according to a separation distance of the diffusion layer from the substrate.
   (24)

A living body attachment-type light emission device according to any one of (1) to (23), further including
a reflection layer that is provided on a surface on an opposite side of a living body attachment side of the flexible light guide plate, reflects the light guided by the flexible light guide plate, and has a plurality of through holes respectively connected to the plurality of through holes of the flexible light guide plate, in which
the substrate is provided at an end portion of the flexible light guide plate, and
a degree of light reflection of the reflection layer changes according to a separation distance of the reflection layer from the substrate.
   (25)

A living body attachment-type light emission device according to any one of (1) to (24), in which
the plurality of through holes of the flexible light guide plate forms a photonic crystal.
   (26)

A living body attachment-type light emission device according to any one of (1) to (25), further including a plurality of protrusion portions formed on a surface on a living body attachment side of the flexible light guide plate.

(27)

A living body attachment-type light emission device according to any one of (1) to (25), further including:
a diffusion layer that is provided on a surface on a living body attachment side of the flexible light guide plate, diffuses the light guided by the flexible light guide plate, and has a plurality of through holes respectively connected to the plurality of through holes of the flexible light guide plate; and
a plurality of protrusion portions formed on a surface on the living body attachment side of the diffusion layer.
   (28)

A living body attachment-type light emission device according to (27), in which
each of the plurality of protrusion portions is formed in such a manner as to extend obliquely with respect to the surface on the living body attachment side of the flexible light guide plate.
   (29)

A living body attachment-type light emission device according to any one of (1) to (28), in which
a plurality of the light emitting units is provided, and
the plurality of light emitting units respectively emits pieces of light having different wavelengths.
   (30)

A living body attachment-type light emission device according to any one of (1) to (29), in which
the flexible light guide plate has a color conversion function of converting a color of the light emitted by the light emitting unit.
   (31)

A living body attachment-type light emission device according to any one of (1) to (30), further including
a diffusion layer that is provided on a surface on a living body attachment side of the flexible light guide plate, diffuses the light guided by the flexible light guide plate, and has a plurality of through holes respectively connected to the plurality of through holes of the flexible light guide plate, in which
the diffusion layer has a color conversion function of converting a color of the light emitted by the light emitting unit.
   (32)

A living body attachment-type light emission device according to any one of (1) to (31), in which
the flexible light guide plate has a function of emitting oxygen.
   (33)

A living body attachment-type light emission device according to any one of (1) to (32), in which
the flexible light guide plate has a function of emitting a photosensitizer.

### REFERENCE SIGNS LIST

1A to 1Q LIGHT EMISSION DEVICE
1Aa LIGHT EMISSION DEVICE
1Ba LIGHT EMISSION DEVICE
1Ca LIGHT EMISSION DEVICE
1Fa LIGHT EMISSION DEVICE
1Ia LIGHT EMISSION DEVICE
1Ja LIGHT EMISSION DEVICE
1La LIGHT EMISSION DEVICE
1Lc LIGHT EMISSION DEVICE
1Ma LIGHT EMISSION DEVICE
1Mb LIGHT EMISSION DEVICE
1Mc LIGHT EMISSION DEVICE
1Pa LIGHT EMISSION DEVICE
10 SUBSTRATE
11 LEAD WIRE
12 POWER SUPPLY
13 CIRCUIT
14 WIRELESS POWER FEEDING UNIT
14a COIL
14b CAPACITOR
15 SENSOR
20 LIGHT EMITTING UNIT
30 FLEXIBLE LIGHT GUIDE PLATE
30a SURFACE
30b SURFACE
31 THROUGH HOLE
35 PROTRUSION ABSORPTION LAYER
40 DIFFUSION LAYER
40a SURFACE
41 THROUGH HOLE
45 PROTRUSION PORTION
50 REFLECTION LAYER
50A REFLECTION LAYER
50B REFLECTION LAYER
51 THROUGH HOLE
60 NON-ADHESIVE LAYER
61 THROUGH HOLE
70 ADHESIVE LAYER
71 THROUGH HOLE
80 RELEASE LAYER
A1 LIVING BODY
A1a TUMOR
B1 ARROW
M1 LIVING BODY ATTACHMENT SURFACE
M2 LIVING BODY NON-ATTACHMENT SURFACE
R1 REGION
R2 REGION
S1 STENT

1. A living body attachment-type light emission device comprising:
a substrate;
a light emitting unit that is provided on the substrate and emits light; and
a flexible light guide plate that is joined to the substrate, guides the light emitted by the light emitting unit, and has a plurality of through holes.

2. The living body attachment-type light emission device according to claim 1, wherein
the substrate is provided at an end portion of the flexible light guide plate.

3. The living body attachment-type light emission device according to claim 1, wherein
the flexible light guide plate is joined to the substrate in such a manner as to cover the light emitting unit.

4. The living body attachment-type light emission device according to claim 1, further comprising
a diffusion layer that is provided on a surface on a living body attachment side of the flexible light guide plate, diffuses the light guided by the flexible light guide plate, and has a plurality of through holes respectively connected to the plurality of through holes of the flexible light guide plate.

5. The living body attachment-type light emission device according to claim 1, further comprising
a reflection layer that is provided on a surface on an opposite side of a living body attachment side of the flexible light guide plate, reflects the light guided by the flexible light guide plate, and has a plurality of through holes respectively connected to the plurality of through holes of the flexible light guide plate.

6. The living body attachment-type light emission device according to claim 1, further comprising:
a first reflection layer that is provided on an outer peripheral side of a surface on a living body attachment side of the flexible light guide plate, reflects the light guided by the flexible light guide plate, and has a plurality of through holes respectively connected to the plurality of through holes of the flexible light guide plate; and
a second reflection layer that is provided on a surface on an opposite side of the living body attachment side of the flexible light guide plate, reflects the light guided by the flexible light guide plate, and has a plurality of through holes respectively connected to the plurality of through holes of the flexible light guide plate.

7. The living body attachment-type light emission device according to claim 1, further comprising:
a diffusion layer that is provided on a surface on a living body attachment side of the flexible light guide plate, diffuses the light guided by the flexible light guide plate, and has a plurality of through holes respectively connected to the plurality of through holes of the flexible light guide plate; and
a reflection layer that is provided on a surface on an opposite side of the living body attachment side of the flexible light guide plate, reflects the light guided by the flexible light guide plate, and has a plurality of through holes respectively connected to the plurality of through holes of the flexible light guide plate.

8. The living body attachment-type light emission device according to claim 1, further comprising
a non-adhesive layer that is provided on a surface on an opposite side of a living body attachment side of the flexible light guide plate, and has a plurality of through holes respectively connected to the plurality of through holes of the flexible light guide plate.

9. The living body attachment-type light emission device according to claim 1, further comprising:
a reflection layer that is provided on a surface on an opposite side of a living body attachment side of the flexible light guide plate, reflects the light guided by the flexible light guide plate, and has a plurality of through holes respectively connected to the plurality of through holes of the flexible light guide plate; and
a non-adhesive layer that is provided on a surface on the opposite side of the living body attachment side of the reflection layer, and has a plurality of through holes respectively connected to the plurality of through holes of the flexible light guide plate.

10. The living body attachment-type light emission device according to claim 1, wherein
the flexible light guide plate has a function of diffusing the light emitted by the light emitting unit.

11. The living body attachment-type light emission device according to claim 1, wherein
the substrate is positioned at an end portion of the flexible light guide plate, and the light emitting unit is provided in such a manner as to emit light toward a surface on a living body attachment side of the flexible light guide plate.

12. The living body attachment-type light emission device according to claim 1, further comprising
a power supply or a wireless power feeding unit that is provided on the substrate and that supplies power to the light emitting unit.

13. The living body attachment-type light emission device according to claim 1, further comprising
a circuit that is provided on the substrate and that is for communication or control.

14. The living body attachment-type light emission device according to claim 1, further comprising
a sensor that is provided on the substrate and that detects a device state or a biological state.

15. The living body attachment-type light emission device according to claim 1, further comprising
a stent around which the flexible light guide plate is wound.

16. The living body attachment-type light emission device according claim 1, further comprising:
a diffusion layer that is provided on a surface on a living body attachment side of the flexible light guide plate, diffuses the light guided by the flexible light guide plate, and has a plurality of through holes respectively connected to the plurality of through holes of the flexible light guide plate; and
an adhesive layer that is provided on a surface on the living body attachment side of the diffusion layer, and has a plurality of through holes respectively connected to the plurality of through holes of the flexible light guide plate.

17. The living body attachment-type light emission device according to claim 1, further comprising
an adhesive layer that is provided on a surface on a living body attachment side of the flexible light guide plate, and has a plurality of through holes respectively connected to the plurality of through holes of the flexible light guide plate.

18. The living body attachment-type light emission device according to claim 1, wherein
a plurality of the substrates and a plurality of the light emitting units are provided, and
the plurality of substrates respectively includes the light emitting units and is respectively provided at both end portions of the flexible light guide plate.

19. The living body attachment-type light emission device according to claim 1, wherein
the flexible light guide plate is formed in a cylindrical shape.

20. The living body attachment-type light emission device according to claim 1, wherein
the substrate is provided at an end portion of the flexible light guide plate, and
a release layer provided on a side of the substrate of the flexible light guide plate is further included.
